(19) United States
(12) Patent Application Publication
(10) Pub. No.: US 2004/0230160 A1
(43) Pub. Date: Nov. 18, 2004

(54) Safety trocar including sealing member

(75) Inventor: Ernesto E. Blanco, Belmont, MA (US)

Correspondence Address: OBLON, SPIVAK, MCCLELLAND, MAIER & NEUSTADT, P.C., 1940 DUKE STREET, ALEXANDRIA, VA, 22314, US

(73) Assignee: Erblan Surgical Inc., Belmont, MA (US)

(21) Appl. No.: 10/791,747

(22) Filed: Mar. 4, 2004

Related U.S. Application Data
(63) Continuation-in-part of application No. 10/092,560, filed on Mar. 8, 2002.
Application No. 10/092,560 is a continuation-in-part of application No. 09/598,453, filed on Jun. 22, 2000, now Pat. No. 6,497,687.
(60) Provisional application No. 60/492,295, filed on Aug. 5, 2003.
(60) Provisional application No. 60/452,105, filed on Mar. 6, 2003.

(51) Int. Cl. A61M 5/178

(52) U.S. Cl. 604/167.06

(57) ABSTRACT

A surgical device includes a sealing member forming a gas tight seal with an instrument disposed in an opening of the sealing member. The sealing member includes a seal ring connected to the interior of the handle, and a conical section having the instrument disposed therein. In an embodiment, the conical section is connected to the seal ring and has a height at least as large as a diameter of a base of the conical section before disposing the instrument therein. In another embodiment, first and second elastic protrusions have the instrument disposed therein, the elastic protrusions contacting one another to form the gas tight seal. In another embodiment, a first section is connected to the seal ring, and a second section is connected to the first section and has the instrument disposed therein. The sealing members, as well as methods of sealing with the sealing members are also disclosed.

### CROSS-REFERENCE TO RELATED APPLICATIONS

This application claims priority to, and is a continuation-in-part of, U.S. application Ser. No. 10/092,560, filed on Mar. 8, 2002, pending, which is a continuation-in-part of U.S. application Ser. No. 09/598,453, filed on Jun. 22, 2000, now U.S. Pat. No. 6,497,687, and claims priority to provisional application No. 60/452,105, filed on Mar. 6, 2003, provisional application No. 60/492,295, filed on Aug. 5, 2003, and provisional application no. (attorney docket no. 249278US17PROV), filed on Feb. 23, 2004, each to Blanco, each of the disclosures incorporated by reference herein in their entireties.

### BACKGROUND OF THE INVENTION

1. Field of the Invention

The current invention relates to a surgical device and, more specifically, to a surgical device containing one or more design features that allow the device to be used safely and effectively.

2. Discussion of the Related Art

Most existing trocars used for endoscopic surgical procedures are incapable of truly effective prevention of injuries to internal organs during insertion and manipulation of the trocar. Despite intensive efforts to improve present trocar designs, the results are still dismal. Present procedures frequently injure internal organs, and the resulting wounds are sometimes serious or even fatal. The need for safer trocars is thus imperative, especially given that endoscopic surgical procedures are likely to become more widespread in the future.

Endoscopic or minimally invasive surgery presents an opportunity to improve present surgical procedures and instrumentation comparable only to the revolutionary effect of the introduction of anesthetics in the 19th Century.

Most present day trocars utilize a tip “shield”, or cover, for the cutting edges which is usually deployed immediately after penetration of the body cavity has taken place. Such a penetration is fraught with danger of injury to internal organs. However careful a surgeon may be during penetration of the body cavity, the resistance to penetration drops at the last instant prior to damage to the internal organs. This sudden drop in the resistance to penetration is called a “plunge effect” and occurs prior to any safety feature deployment. In some trocars, the penetration is controlled in some fashion, either taking place in small increments or under some form of approximate direct observation, estimate, or monitoring. In all cases, however, the designs result in much of the piercing tip being inserted to a dangerous depth before any protecting devices is deployed. This is perhaps not surprising since, after all, a hole must be made before any protection is deployed.

Since in most cases delicate organs are very close to the inside of the skin layer being pierced, it is advisable to perform the penetration after internal cavities have been filled with carbon dioxide to minimize the danger of accidental injury due to contact with the sharp piercing tip or the cutting edges of the instrument. In most cases, however, the force required for penetration and the elastic nature of the muscular layer cause a severe depression at the surgical portal, therefore bringing the penetrating tip of the instrument closer to the internal organs. In some of those cases, the sudden penetration of the cavity wall and the rapid drop in resistance allow the instrument to be propelled far deeper than desired or is possible to control. Furthermore, friction between the tissue walls and any protective device retards the deployment of the protective device, and an injury almost inevitably occurs.

After the body cavity is penetrated, the trocar must be sealed to prevent gas flow from leaking from the body cavity. Undesired gas flow from the body cavity should be prevented during insertion, manipulation, and removal of various instruments relative to the trocar. Currently, a separate flap valve and seal are used—the seal preventing gas flow between the trocar and the instrument disposed therein, and the flap valve preventing gas flow after the instrument has been withdrawn from the trocar. However, manufacture and assembly of the trocar including the multiple components of the flap valve and seal are complicated and expensive. Further, the seal is not always effective in preventing undesired gas flow from the body cavity for various instruments having various diameters, when the instrument is misaligned in the seal, when the instrument is manipulated through a large range of movement, and when the instrument is moved laterally or axially within the seal. The conventional seals shown in U.S. Pat. Nos. 5,209,737, 5,308,336, and 5,385,553 do not adequately solve these problems.

### SUMMARY OF THE INVENTION

Accordingly, one object of this invention is to insure that such events are avoided through a surgical device in which a penetrating tip or cutting edge(s) of the instrument be kept, at all times, sufficiently distant from delicate tissues. Thus, even under dynamic conditions, the probability of injury will be reduced.

A further object of this invention is to provide a surgical device wherein insufflation fluid can be driven into a patient during penetration of the body cavity by the surgical device to drive the internal organs away from the surgical device during penetration. The insufflation fluid of the present invention can either be supplied from an external pressurized reservoir, or compressed (and hence gathered) during penetration of the body cavity by the surgical device.

A further object of the invention is to provide a surgical device that contains one or more cutting edge that provides low frictional forces between the cutting edge and tissue during penetration of the body cavity, thus reducing the force needed to drive the surgical device into the body cavity.

A further object of the invention is to provide a surgical device that includes a protective device that deploys while remaining substantially out of contact with tissue, thus reducing frictional forces between the protective device and ensuring a controlled and advantageous deployment.

A further object of the invention is to provide a surgical device that includes a protective device such as safety guards, wherein the guarding elements have an apex and the angle subscribed at the apex is smaller than the angle subscribed by the blades or cutting elements of the surgical device, thus insuring progressive coverage of the blades or cutting elements during deployment of the protective device.

A further object of this invention is to provide a surgical device with a grip mechanism that allows convenient gripping and twisting of the surgical device during penetration of the body cavity.

A further object of this invention is to provide a surgical device that includes a locking system that prevents accidental reuse of the cutting elements after the tip has been used.

A further object of the invention is to obviate disadvantages of known sealing configurations for the trocar, including obviating the need for separate flap valves and seals.

It is therefore desired that this invention, in general, improve surgical safety.

These and other objects can be provided by a surgical device including a handle configured to be gripped, a cannula connected to the handle, and a sealing member disposed in an interior of the handle and configured to form a gas tight seal with an instrument disposed in an opening of the sealing member. In an embodiment of the invention, the sealing member includes a seal ring connected to the interior of the handle, and a conical section configured to have the instrument disposed therein, the conical section connected to the seal ring and having a height at least as large as a diameter of a base of the conical section before disposing the instrument therein. In another embodiment of the invention, the sealing member includes a seal ring connected to the interior of the handle, a conical section configured to have the instrument disposed therein, the conical section connected to the seal ring, and first and second elastic protrusions configured to have the instrument disposed therein, the elastic protrusions configured to contact one another to form a gas tight seal.

The present invention further provides a surgical device including means for forming a gas tight seal between with an instrument removably disposed therein, the means for forming the gas tight seal having a height at least as large as a diameter of a base of the means for forming the gas tight seal before disposing the instrument therein.

The present invention still further provides a surgical device including means for forming a gas tight seal between with an instrument removably disposed therein and for forming a gas tight seal between portions of the means for forming the gas tight seal when no instrument is disposed therein.

The present invention still further provides a method of sealing a surgical device, including forming a seal between an instrument and a sealing member, the sealing member having a height at least as large as a diameter of a base of the seal when the instrument is not disposed in the seal.

The present invention still further provides a method of sealing a surgical device including disposing an instrument in a conical member, and forming a seal between protrusions connected to the conical member and the instrument.

The present invention still further provides a method of sealing a surgical device including disposing a sealing member in an interior of a handle, and forming a gas tight seal with an instrument disposed in an opening of the sealing member. The sealing member includes a seal ring connected to the interior of the handle, and a conical section configured to have the instrument disposed therein, the conical section connected to the seal ring and having a height at least as large as a diameter of a base of the conical section before disposing the instrument therein.

The present invention still further provides a method of sealing a surgical device including disposing a sealing member in an interior of a handle, and forming a gas tight seal with an instrument disposed in an opening of the sealing member. The sealing member includes a seal ring connected to the interior of the handle, a conical section configured to have the instrument disposed therein, the conical section connected to the seal ring, and first and second elastic protrusions configured to have the instrument disposed therein, the elastic protrusions configured to contact one another to form the gas tight seal.

The present invention still further provides a surgical device including a sealing member disposed in an interior of the handle and configured to form a gas tight seal with an instrument disposed in an opening of the sealing member. The sealing member includes a seal ring connected to the interior of the handle, a first section connected to the seal ring, and a second section connected to the first section and configured to have the instrument disposed therein.

In a preferred embodiment, the surgical device includes a valve configured to form a gas tight seal when no instrument is disposed therein.

The present invention still further provides a sealing assembly including a seal ring configured to be connected to an interior of the device, a first section connected to the seal ring, and a second section connected to the first section and configured to have the instrument disposed therein.

In a preferred embodiment, the sealing device includes a valve configured to form a gas tight seal when no instrument is disposed therein.

The present invention still further provides a method of sealing a device including disposing a sealing member in an interior of the device, the sealing member configured to form a gas tight seal with an instrument disposed in an opening of the sealing member. The sealing member includes a seal ring connected to the interior of the handle, a first section connected to the seal ring, and a second section connected to the first section and configured to have the instrument disposed therein.

In a preferred embodiment, the method of sealing includes disposing a one way valve in an interior of the device, the valve configured to achieve a gas tight seal when no instrument is disposed in the valve.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 shows a general view of an example trocar in isometric pictorial form;

FIG. 2 illustrates a partial broken view of the penetrating end of the example trocar with guards removed to behind the tip knives to illustrate a shape of this embodiment more clearly;

FIG. 3 shows the same end of the example trocar with the guards installed but retracted as when penetration of an example embodiment starts, and thus, the knife edges are exposed and ready to start cutting;

FIG. 4 shows the tip of the guards protruding ahead of the cutting tip as when the tip had just started to pierce the abdominal cavity;

FIG. 5 shows the tip of the example trocar with the guards fully extended and covering the knife edges as when completely inside of the abdominal cavity;

FIG. 6 shows the example trocar tip at the moment it approaches the skin layer, and thus the guard tips are beginning to push against the skin and be retracted into the penetrator;

FIG. 7 illustrates the point when, in an example embodiment, the guards are completely pushed into the retracted position and the knife tips start to cut into the tissue;

FIG. 8 illustrates the point when, in an example embodiment, the knife tips have completed the passage across the tissue and begin to emerge across the endothelial layer into the abdominal cavity, and thus the tips of the guards begin to push into the incipient opening while a forceful jet of pressurized carbon dioxide gas pushes delicate internal tissues away from the immediate penetration region;

FIG. 9 illustrates the point when, in an example embodiment, the tips of the guards have penetrated the opening and prevent any contact between the knife tips and the surrounding internal tissues while the exposed knife edges behind the opening continue the cutting action, and the pressurized carbon dioxide gas expansion continues to hold delicate tissues away from the cutting region;

FIG. 10 illustrates, in an example embodiment, the continuing penetration, and thus the guards have penetrated almost completely, while behind them the still-exposed edges continue the cutting action and the passage of gas continues;

FIG. 11 illustrates the point in an example embodiment when the penetration has been completed. The knife edges are fully covered by the guards and the tissue opening allows for the passage of the cannula and the insufflation continues until completed and the penetrator assembly can be removed;

FIG. 12 shows the top view of an example trocar handle with a portion broken away to show some internal details;

FIG. 13 illustrates a longitudinal section along a vertical plane “A-A” to exhibit most of the internal details of an example trocar handle;

FIG. 14 illustrates a top view of the distal section of an example handle with the grasping horns to facilitate manipulation;

FIG. 15 illustrates an end view of the distal section of an example handle as seen from the right showing also a partial broken section detail of the flap valve pivot and lever;

FIG. 16 illustrates a partial isometric view of the example locking mechanism for the guards stem showing some of the elements within the proximal section of the handle as in Section “A-A” on FIG. 13;

FIG. 17 illustrates an exploded view of some of the example elements of the guards stem locking mechanism in an example spatial relationship;

FIG. 18 illustrates an example locking mechanism in a locked position;

FIG. 19 illustrates an example locking mechanism having been unlocked and ready for the start of penetration;

FIG. 20 illustrates how pushing the guards against the skin has forced their stem towards the right;

FIG. 21 illustrates a position of the stem where the guards are completely retracted and the knife edges filly exposed for cutting;

FIG. 22 illustrates a position of the locking mechanism after the full release of the guards into the abdominal cavity and the locking of their stem back to its initial position shown in FIG. 18;

FIG. 23 shows an isometric view of an embodiment of a sealing member in an uninstalled and unstretched or undeformed state;

FIG. 24 shows a front view of the embodiment of the sealing member of FIG. 23;

FIG. 25 shows a side view of the embodiment of the sealing member of FIG. 23;

FIG. 26 shows a top view of the embodiment of the sealing member of FIG. 23;

FIG. 27 shows a bottom view of the embodiment of the sealing member of FIG. 23;

FIG. 28 shows a front view of the embodiment of the sealing member of FIG. 23 in an installed and stretched or deformed state;

FIG. 29 shows a bottom view of the embodiment of the sealing member of FIG. 28;

FIG. 30 shows a top view of the embodiment of the sealing member of FIG. 28;

FIG. 31 shows a side view of the embodiment of the sealing member of FIG. 28;

FIG. 32 shows an isometric view of an embodiment of a sealing member in an uninstalled state;

FIG. 33 shows a front view of the embodiment of the sealing member of FIG. 32;

FIG. 34 shows a top view of the embodiment of the sealing member of FIG. 32;

FIG. 35 shows a bottom view of the embodiment of the sealing member of FIG. 32;

FIG. 36 shows a front view of the embodiment of the sealing member of FIG. 28 in an installed state;

FIG. 37 shows a bottom view of the embodiment of the sealing member of FIG. 36;

FIG. 38 shows a top view of the embodiment of the sealing member of FIG. 36;

FIG. 39 shows an isometric view of a maximum diameter instrument partially disposed in an embodiment of the sealing member;

FIG. 40 shows an isometric view of the maximum diameter instrument further disposed in the sealing member of FIG. 39;

FIG. 41 shows an isometric view of the maximum diameter instrument fully disposed in the sealing member of FIG. 39;

FIG. 42, shows an isometric view of a minimum diameter instrument disposed in the sealing member of FIG. 39;

FIG. 43 shows an isometric view of a relatively larger diameter instrument disposed in an embodiment of the sealing member;

FIG. 44 shows an isometric view of a relatively smaller diameter instrument disposed in the sealing member of FIG. 43;

FIG. 45 shows an isometric view of the relatively smaller diameter instrument disposed in an orientation in the sealing member of FIG. 43;

FIG. 46 shows an isometric view of the relatively smaller diameter instrument disposed in another orientation in the sealing member of FIG. 43;

FIG. 47 shows an isometric view of the relatively smaller diameter instrument being withdrawn from the sealing member of FIG. 43;

FIG. 48 shows a cross sectional view of an embodiment of a sealing member and a valve;

FIG. 49 shows an isometric view of the valve of FIG. 48;

FIG. 50 shows a cross sectional view of a minimum diameter instrument disposed in the sealing member of FIG. 48; and

FIG. 51 shows a cross sectional view of a maximum diameter instrument disposed in the sealing member and the valve of FIG. 48.

### DESCRIPTION OF THE PREFERRED EMBODIMENTS

Referring now to the drawings, wherein like reference numerals designate identical or corresponding parts throughout the several views, and more particularly to FIG. 1 thereof, wherein a cannula 2 is firmly attached to a distal section of a handle which is formed from two segments, the distal one 6 externally containing gripping horns 6a, insufflation device 11, and flap valve lever 12, and a proximal handle section 5 in the shape of a hemispherical knob to facilitate its pushing with the palm of the hand. This section also contains a depression 9 with a flat bottom 9a, and external mechanisms including a button 7 inserted for sliding into a slot 8 to monitor and control the position of safety guards at the extreme distal end of cannula 2. The safety mechanisms protruding distally from cannula 2 include conical tissue expanders 4, and safety guards 3 intended to cover a set of knives (not visible in this FIG. 1). Those are the externally visible features of this invention.

FIG. 2 shows details at the penetrating distal end of the trocar. A hollow outside cylinder 2 is the cannula which is firmly attached to the distal section of the handle 6 as was described in FIG. 1. Inside of the cannula 2, there is another hollow cylinder 13 which is the penetrator. This is the removable part which is attached to the proximal section of the handle 5, and can be removed after the penetration is completed to allow for the introduction of surgical instruments. The cannula 2 has its distal end beveled as shown by 2a to facilitate its introduction across the tissue opening with minimal resistance. The penetrator hollow cylinder 13 has its distal end formed as a plurality of conical segment expanders 4 which are spaced by slots 4a to allow for the protrusion of pointed flat knives 14 joined at the center of the instrument and resembling thin arrowheads joined at a center. As shown in FIG. 2, the knives are positioned into the penetrator hollow cylinder 13 to a depth shown at 14a. The knife edges outside the slots 4a between the conical segment expanders protrude a substantial distance to insure adequate cutting. The set of knives is assembled into the penetrator cylinder 13 by spot welds 15, or by other similar mechanism. Right behind the crossing of the knife blades can be seen the plastic guard tips 3a. In FIG. 2, the guards are shown as removed from the knives so as to facilitate the understanding of their shapes and relationship to the knives. The subassembly of the guards 3 is part of a support disk 16 which in turn is part of the guards hollow stem 17 connecting them to an actuator spring and locking mechanism at the proximal section of the handle (not shown here). In the real instrument, the guard tips 3a are inserted around the knife blades which fit into the narrow spaces 3b between the guards. The guards are then assembled by being pushed forward until they protrude between the blade sides and the conical expander slots 4a as can be shown in FIG. 3 below. In FIG. 3, the tips of the guards are barely visible because the guards are retracted as when the trocar is first pushed against the skin.

FIG. 4 shows the tips of the guards 3a protruding ahead of the tip of the knives and covering them. A short distance behind the tips of the guards 3a the edges of the knives 14 are exposed and capable of cutting. FIG. 4 shows the configuration of the trocar cutting tip right after initiation of the penetration across the abdominal tissue. At that instant, the guard tiny tips 3a plunge across the start of the opening and quickly cover the sharp cutting point while the exposed knife edges continue cutting inside the skin until the penetration is complete as shown in FIG. 5. FIG. 5 shows how the front end of the example trocar looks after the penetration into the abdominal cavity has been completed. At that time all edges of the cutting knives are covered by the fully extended guards and the whole penetrator assembly can be pulled out with the proximal sector of the handle.

As will be shown later, in one embodiment, at the instant when the first perforation of the abdominal wall was made, a forceful jet of carbon dioxide gas issued across the perforation to deflect away any delicate organs close to the knives tip while simultaneously the guard tips entered the opening to cover the point of the knife edges. The operations just described above are a critical part of this invention, therefore they will best be described through the sequence of figures from FIG. 6 through to FIG. 11.

FIG. 6 represents the example trocar guard tips 3a as they begin to contact the skin layer 20. The internal organs are shown at the left side as 25. At this instant, the skin outside layer is deflected under the force of the guard tips which are urged forward by their spring. As the trocar is pushed forward, the guards will be forced into the penetrator 13 and displace the base disk 16 and guard stem 17 toward the right against the force of their spring.

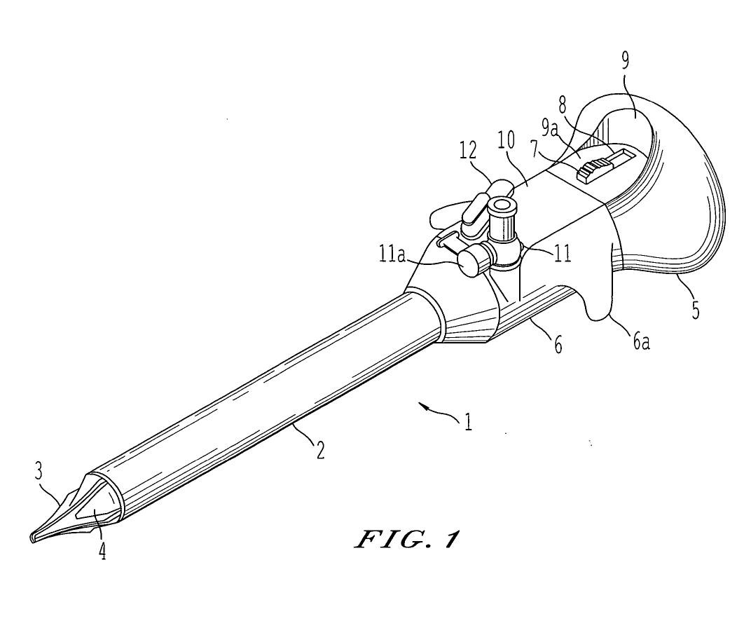

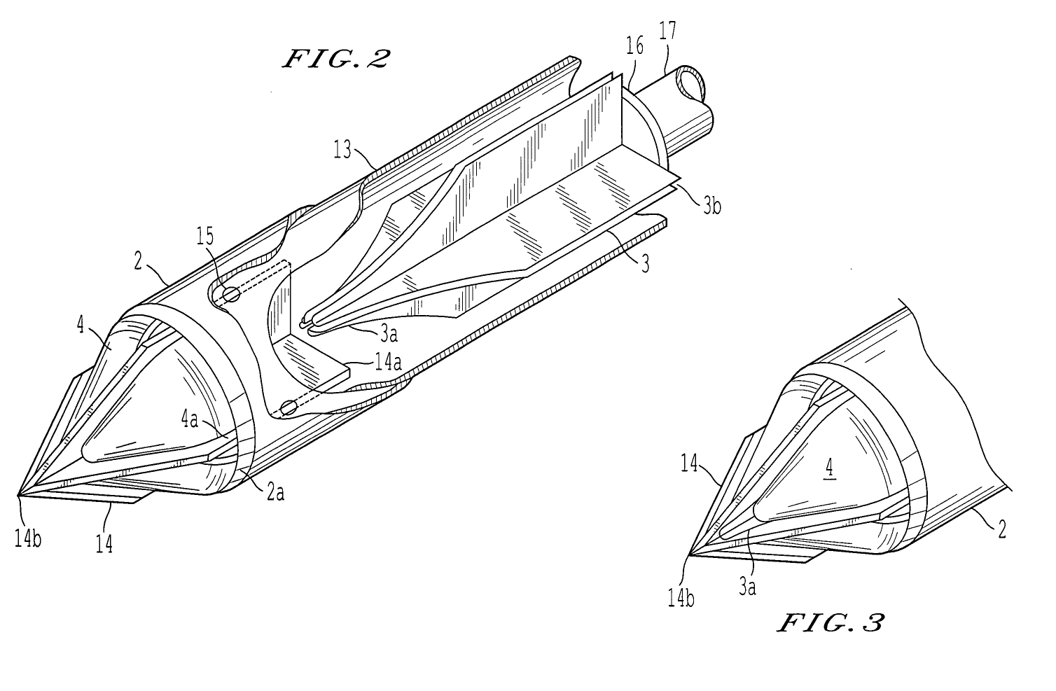

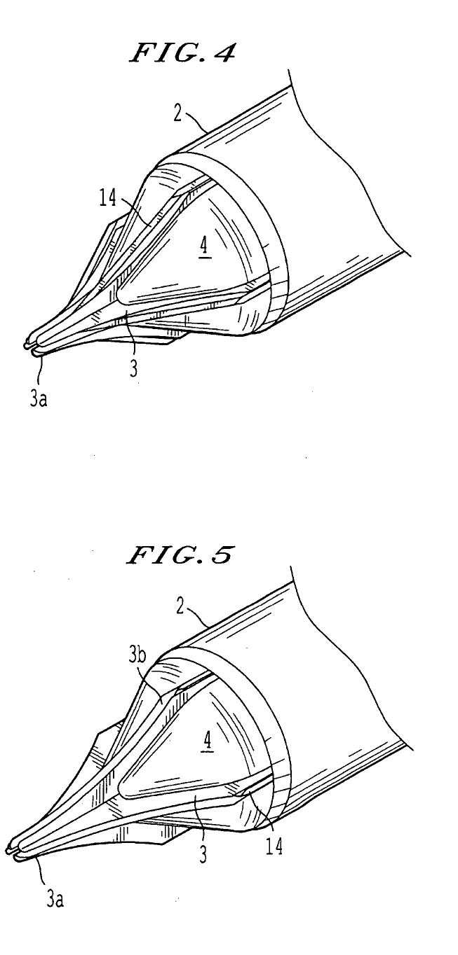

FIG. 7 shows the guards 3 already completely retracted into the penetrator 13, and the knife edges 14 completely exposed. At that instant, the point of the knives begins to cut and penetrate at 21 into the outside tissue layer. As shown in FIG. 7, the cutting pathway of the cutting tip/knife edge is of a smaller diameter than the inner diameter of the cannula 2 such that the cut made by the blade results in a smaller lumen or bore than that of the cannula. At that time, the carbon dioxide gas is allowed to pressurize the inside of the penetrator 13, and while some gas may escape at first, the tissues around the tip will seal the flow until the cutting tip starts to emerge across the internal abdominal wall.

FIG. 8 shows the onset of penetration. At that instant, the cutting tip point 14b has made a very minute perforation 23 and, because of the presence of the guard tips 3a, there is enough space to allow a fluid flow (shown here as a gas jet 24) to issue out and cause the displacement of nearby internal organ tissues 25a, while simultaneously the guard tips 3a expand the opening urged by their spring pushing at 17 and plunge through the perforation effectively covering the cutting tip 14b.

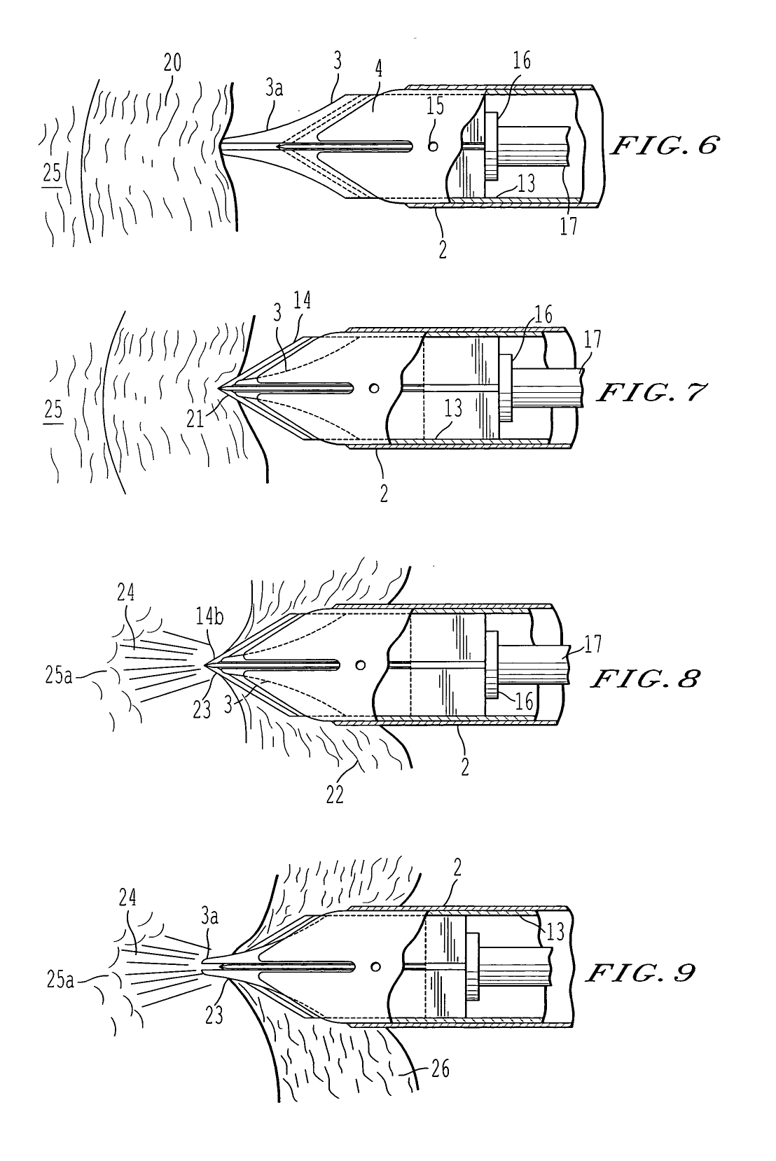

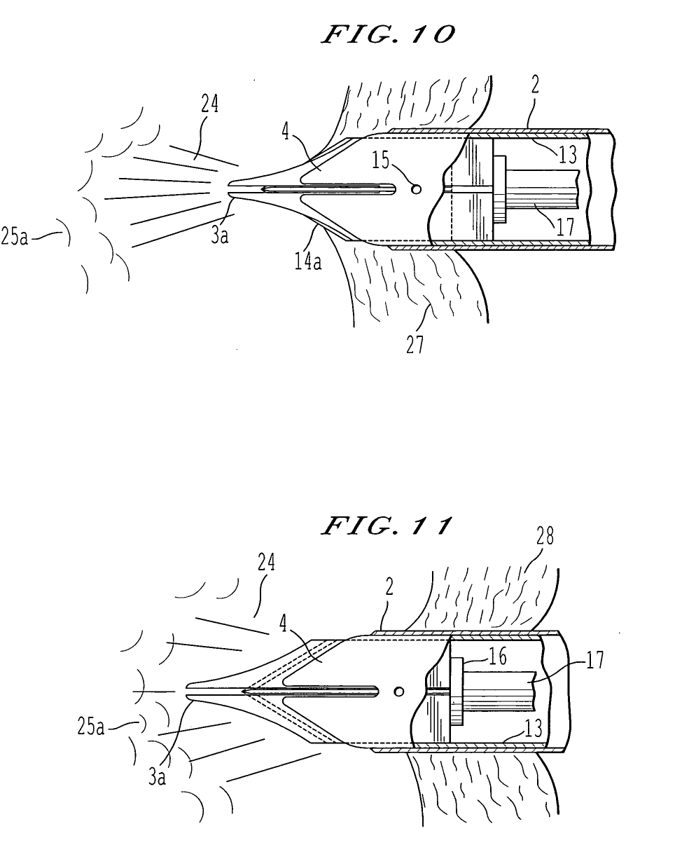

FIG. 9 shows the result of the action described above. The gas jet 24 continues issuing and driving internal organs 25a farther away while the guard tips 3a completely enclose the cutting tip 14b. All danger to internal tissues has passed. The extremely quick flow of the gas and the action of the guard tips make the manipulation factors of this trocar the safest to master easily. The force or speed of the penetration action are, within reason, almost immaterial.

FIG. 10 shows the penetration process. The cannula 2 is partly introduced across the tissue 27 and the guard tips 3a continue advancing and protecting the internal tissues from the knife edges while the portions of the edges not yet covered by the guards 14a are seen cutting the remainder of the opening ahead of the cannula, and the tissue expanders 4 facilitate penetration by protecting the guards from tissue friction. At this point of the penetration the flow of carbon dioxide gas 24 is fairly unimpeded and performs the insufflation stage of the process, driving internal organs 25a farther away from the trocar portal.

FIG. 11 shows the trocar after full insertion and in the last stage of insufflation. The knife edges are now fully covered by the guards, and the cannula 2 is seen fully inserted across the tissue. The insufflation continues until completed and then the penetrator 13 is removed to allow the insertion of surgical instruments across the cannula.

Having described in sequential detail the insertion, guarding, and insufflation operations, and the mechanical parts that perform them it remains to describe the additional way by which all that is accomplished. The mechanisms that allow this are located in the handle of the instrument.

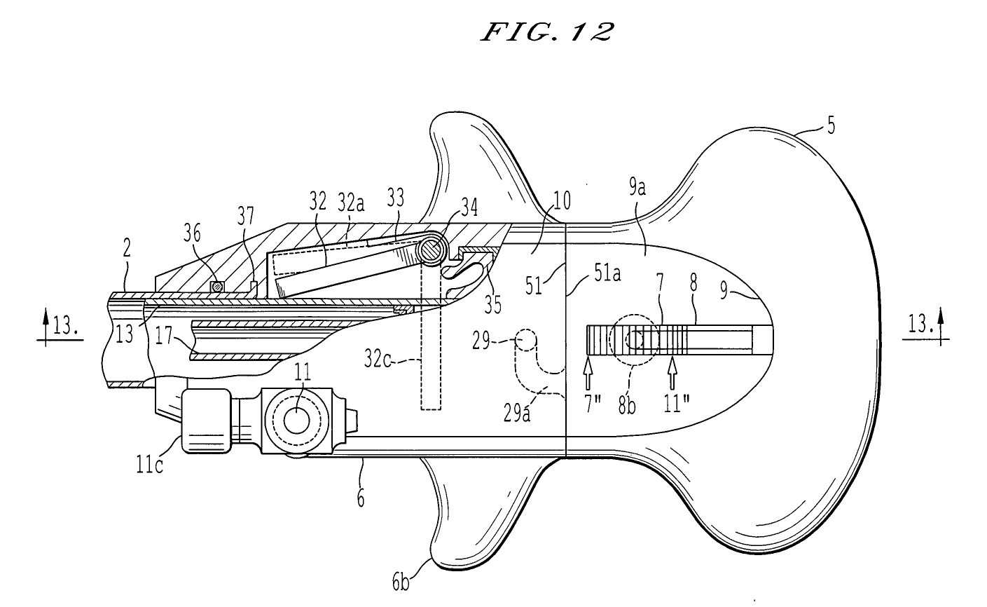

FIG. 12 is a top view of the trocar showing some of the external parts as well as a partial broken view of some interior parts. The body of the handle is made out of plastic and has two main segments. The proximal segment 5 is designated to fit into the palm of the hand and has a proximal end of hemispherical shape with a depression of arcuate profile 9 at the top terminating at a flat surface 9a where the guard stem controls are located. Those controls are recessed into the flat depression 9a to prevent unwanted actuation, and include a double slot with vertical slots 8 and 8a into which is inserted a button 7 and its rectangular guiding shank 7a. The button 7 is capable of vertical and horizontal movement, the latter movement being limited between arrows 7b and 7c as will be described later. The proximal segment 5 is assembled as an integral part of the penetrator system. Its distal end 51 forms the interface between the two segments of the handle.

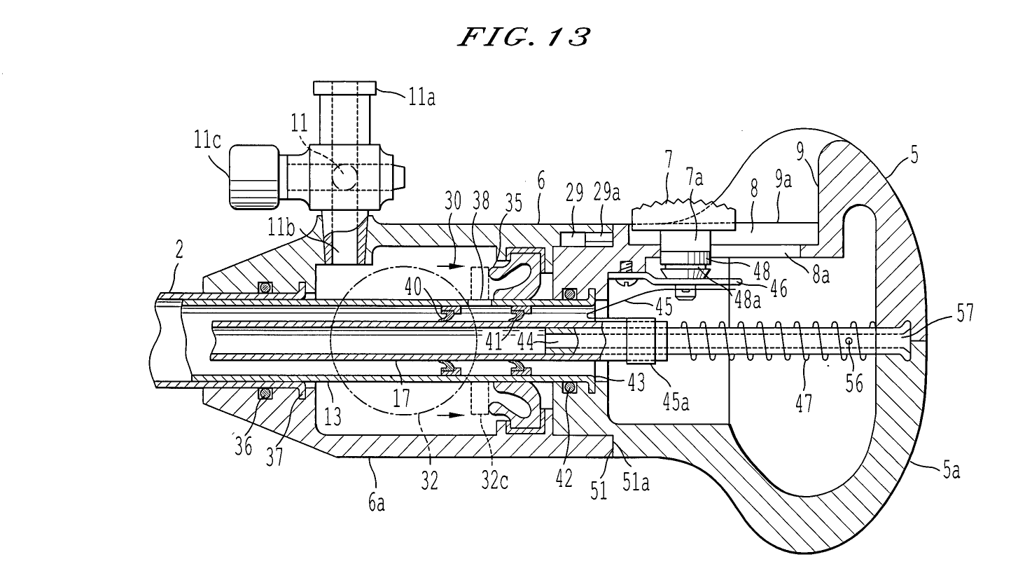

The distal segment 6 of the handle has two lateral protruding horns 6b to facilitate its manipulation during penetration and orientation. The two handle segments 5 and 6 are locked together during usage by way of a bayonet stud 29 and slot 29a. During insertion the stud 29 on part 5 is aligned with the slot 29a on part 6, pushed, and turned clockwise, until the stud locks the two segments firmly, the knob on 5 and the horns 6b provide a good grasp for that operation. The slot 29a has a slant at the transversal direction running slightly away from the interface 51 so as to insure that the turning-locking motion will assure a firm and stable connection. This will be discussed further in reference to FIG. 14.

The partial broken section at the top left of the distal segment 6 is intended to show the operation of the flap valve 32, which acts as a check valve in the illustrated embodiment. The valve has a shaft 34 pivoted between the upper 6 and lower 6a portions of the handle and is urged to rotate counterclockwise by a torsional spring 33 located around the shaft 34. The shaft of the flap valve is firmly attached to the valve and can be rotated from outside the body segment 6 as will be shown later on FIG. 14. An external lock allows the valve to remain open during desufflation if turned hard to its stop position 32a shown in dotted lines. As shown in the embodiment illustrated in FIG. 12, the valve has been opened by the insertion of the penetrator 13. In other cases, the valve could be opened for surgical or visualization instruments. When left to itself, the valve will turn counterclockwise and snap shut against the face of seal 35 which serves as face seal for the valve and lip seal for the penetrator 13. The left end of FIG. 12 shows how the cannula 2 is attached to the handle segment 6 by way of a flange 37, and prevented from leaking by an “O” ring 36. In the same FIG. 12 is shown how the carbon dioxide gas spigot manual valve 11 is mounted at one side of the top of segment 6.

FIG. 13 is a longitudinal vertical cross section along a plane “A-A” to show the internal details of the handle. As can be noticed, the two segments of the handle include a top and a bottom part split along a horizontal plane for fabrication, one becoming 5 and 5a, and the other 6 and 6a, and after each segment has been fitted with the internal parts at assembly the two halves of each segment are permanently bonded together. Each of the two segments is assembled separately since they must be detached and attached during usage. The penetrator segment is only used to make the entry portal, but it must be emphasized that it is such step that involves the greatest risk.

The distal segment made of parts 6 and 6a houses the cannula 2 and all the gas infusion and valving. The connection of the cannula to the segment part 6 was described before. FIG. 13 shows the gas connector or layer 11a to which the gas line is affixed. The valve system is bonded via a conical stem 11b into a boss on plane 10 so the incoming gas flows in the direction of arrow 30 and pressurizes the space between the inlet and the seal 35 from where it can enter the openings 38 around the penetrator 13 walls and fill the space between lip seals 40 and 41. Since the lip seals are oriented toward the front the pressure will open lip seal 40 but not lip seal 41 and the gas will fill and pressurize the entire space along the penetrator 13, not being able to escape when the trocar tip has been inserted into the tissue, however, as soon as the smallest opening is made by the point of the blades the gas will escape as a jet and deflect the surrounding internal organs away from the entry portal. Lip seal 40 is intended to prevent back flow from the penetrator in case of accidental opening or leakage across the gas valve during a procedure. In such a case, the pressurized volume of gas within the penetrator 13 will suffice to insure the safe deflection of nearby tissues even before the tips of the guards 3a plunge into the opening. The guards stem 17 is completely sealed at the front by disk 16 and thereby its interior can be at atmospheric pressure, however, since it must slide back and forth with the guards it must also be supported at the proximal end and must be guided over a stationary hollow steel stud 44 inserted into it to a minimal depth of four diameters. The proximal end of stud 44 is flared to provide fixation between parts 5 and 5a of the proximal hemispherical knob. A hole 56 on the hollow stud 44 serves to provide air passage in and out of the stud when the guards stem moves back and forth acting as a piston pump. The hole 56 should pass through the stud and be of a diameter such as not to impede flow and dampen the sliding action of the guards' stem. Compression coil spring 47 mounted around stud 44 serves to provide the required force to urge the guards stem in the distal direction. The proximal end of the penetrator outside cylinder 13 is flared at 43 for fixation onto the proximal handle segment parts 5 and 5a. It is also sealed at the front by an “O” ring 42 to insure that no leakage of gas would occur even if seal 35 should leak: flared tubular assemblies like 43 are not reliable seals.

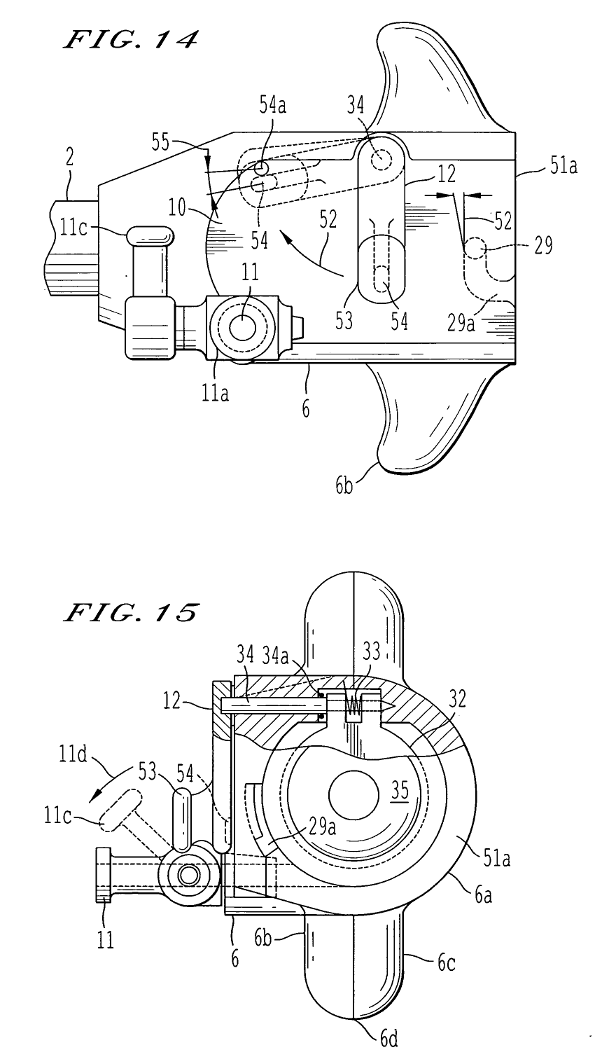

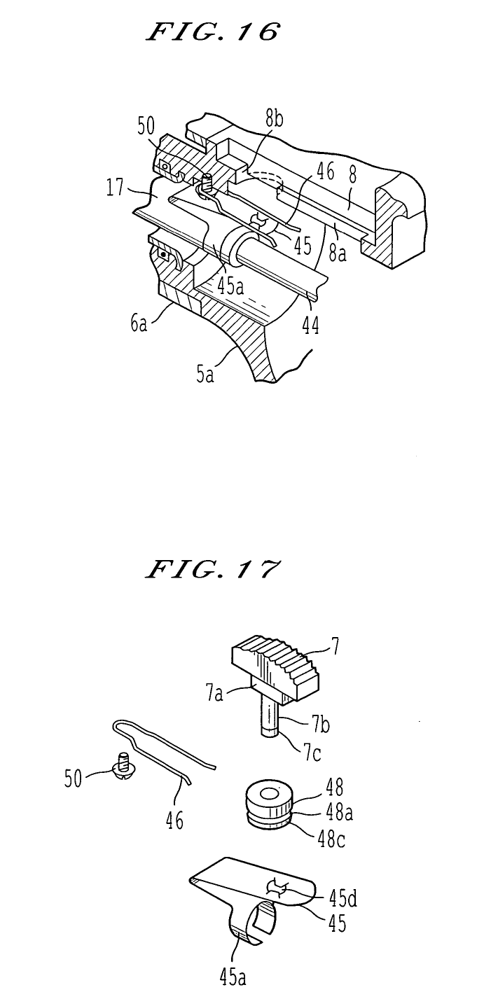

The proximal handle segment formed by 5 and 5a is attached to the penetrator 13 and contains all its functional and control elements. The guard stem 17 has at its proximal end a shallow cylindrical depression into which a thin ring 45a which is part of leaf spring 45 is affixed. The exact configuration of the locking system to which the spring 45 belongs can be seen in FIGS. 16 and 17, and its function in the sequence of FIGS. 18 through 22. FIG. 17 is an exploded view of some of the elements of the locking system in their proper relationship. At assembly, the button 7 is inserted across slot 8 on the top surface 9a on FIG. 13 and the locking cylinder 48, which has a circumferential groove 48a and a conical end 48c is pushed up along the stem 7b against the bottom of the rectangular guide 7a thereby assembling button 7 into the slot 8a. As the assembly continues the lower tip of stem 7b is pushed hard against the punched hole 45d of the leaf spring until groove 7c is gripped by the lateral tabs at 45d and the assembly of the button is complete. If now the open hollow cylinder 45a is snapped onto the surface depression at the proximal end of stem 17, the button 7 becomes axially fixed to stem 17 and will follow its back and forth motion in response to coil spring 47 and the forces at the tip of the guards. FIG. 16 shows the assembly of the U spring 46 to the lower inside of 5 by the use of screw 50. FIG. 16 does not show button 7 for the sake of clarity, but it shows flat spring 45 pushing up against the bottom of the U spring 46. If the assembly of the button 7 and the locking cylinder 48 was shown there, it would be evident that the button would be pushed upwards and the locking cylinder 48 would be forcibly inserted into the round socket 8b, thereby preventing any motion of the flat spring 45 and the guards stem 17 attached to it by ring 45a. That is the situation depicted on FIG. 13.

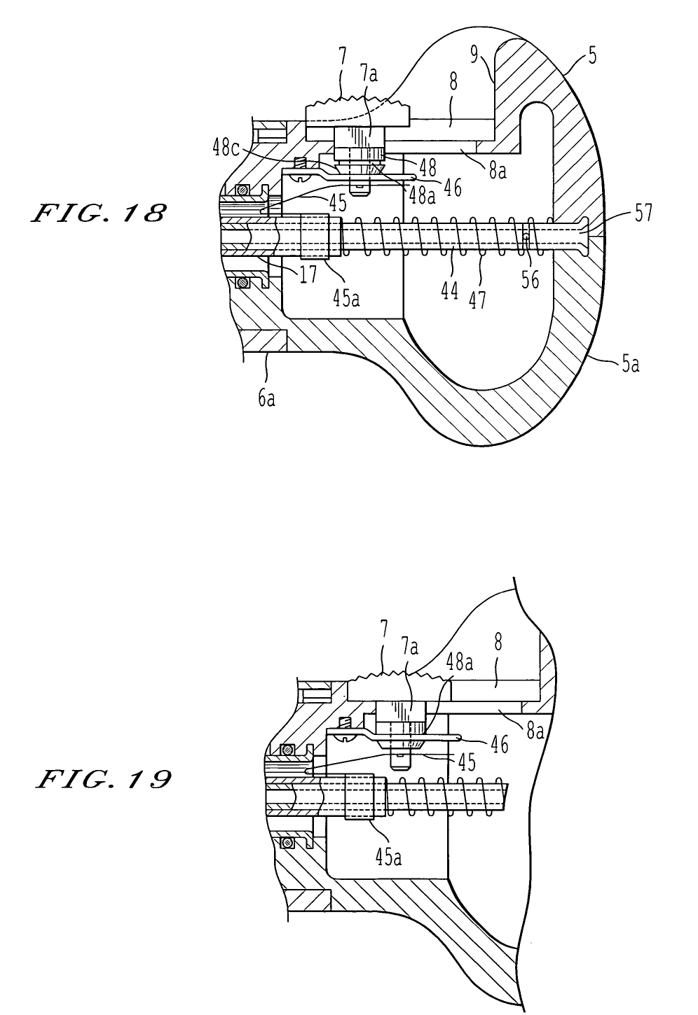

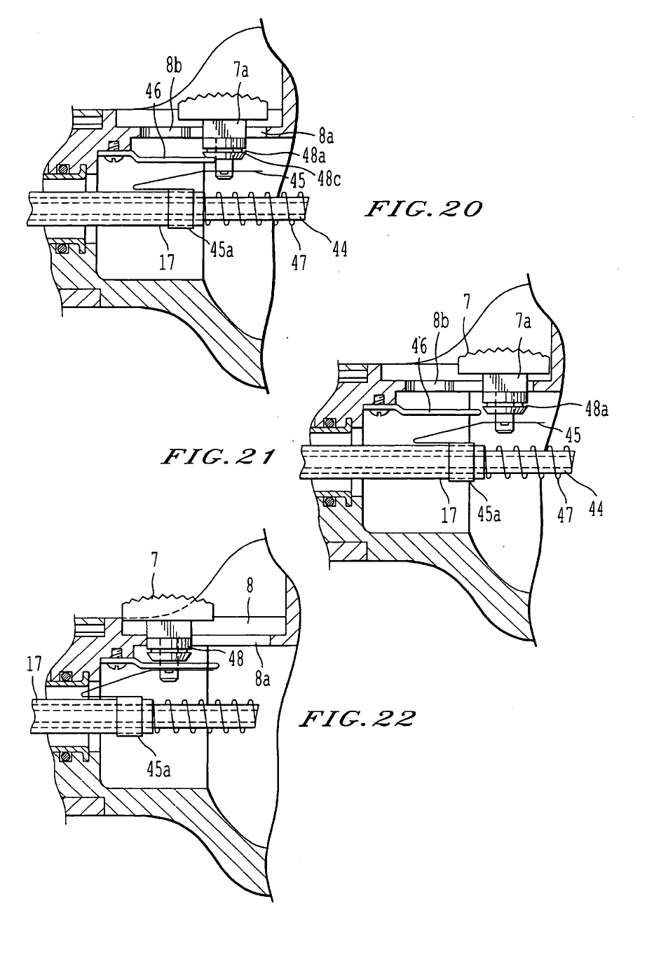

FIGS. 18 through 22 describe an operation of an example locking system in detail, as follows. In the position illustrated in FIG. 18 the system is locked: the guards stem and the guards cannot move at all since the cylinder 48 is inserted into the round socket 8b. FIG. 19 shows what happens when button 7 is pushed down. When that is done the conical end 48c of cylinder 48 opens the U spring 46 and the spring then snaps close into the groove 48a thereby disengaging the locking cylinder from the round socket 8b. The system is then unlocked. The trocar is said to be “armed”, and able to permit the motion of the guards backwards, exposing the cutting blades for penetration of the skin. That is the position depicted on FIG. 6. The following discussion is directed to the embodiment shown in FIG. 20. The penetrating force against the skin pushes on the guards and the guards stem 17, and the connecting flat spring 45 moves the button 7 proximally. The rectangular slide section 7a enters the space between guides 8a, and soon afterwards, the locking cylinder groove 48a disengages from the open end of the U spring 46, and the spring 45 pushing upwards against the stem groove 7c forces the top of the locking cylinder to snap against the underside of the groove 8a. In that position, the locking cylinder 48 is free to continue sliding along the underside of groove 8a as shown in FIG. 21 until the initial penetration is made and the force of the coil spring 47 urges the guards stem 17 and the flat spring 45 to return the button 7 to its initial position, at which time the locking cylinder will pass freely over the U spring 46 and snap back into the round socket 8b locking the system into the “safe position” where the guards cannot move accidentally. FIG. 22 shows the completion of the cycle back to the initial configuration of FIG. 18.

A quick review of the provided example locking system from the user viewpoint reveals that the operations include “arming” the trocar by pushing down on the button at the top of the handle at position 7′ shown in FIG. 12, until it “snaps” down; then pushing the trocar against the skin and watching or listening to the position of the button as it slides towards 7′ and then “snaps” to its initial position 7′. That will be the indication of having completed the penetration. If, for any reason, button 7 were pushed down accidentally, it could be reset to the “safe” condition by merely moving it in the direction to 7′ and then releasing it. It should then get snap-locked at a high level in position 7′, and could not be moved without first pushing it down.

The details of operation of the example flap valve, its design, and locking for deflation are seen in FIGS. 14 and 15. FIG. 14 shows the top view of the handle distal segment, previously presented in FIG. 12 as a partial broken section to show the interior details. FIG. 14, however, is intended to show the external operative controls on this segment of the handle in the interest of the user. The flap valve lever 12 is shown in the closed position as it should be when the penetrator is removed. The lever is attached to a shaft 34 whose opposite end is attached to the flap 32 as seen in FIG. 15. The insertion of the internal trocar elements is performed when the top 6 and bottom 6a of each handle segment are separated prior to their being bonded along plane 6d.

FIG. 15, as explained before, is the end view of the example embodiment previously illustrated in FIG. 14 as seen from the right side. That is how the distal segment of the handle will appear when the proximal segment is removed. The flap valve external lever knob 53 is provided with a small depression 54 at its bottom to allow it to be held open when the depression is forcibly made to engage a small knob 54a protruding from the flat surface 10 after the lever has been turned in the direction of arrow 52. That is the desufflation position of the valve which allows the surgeon to use both hands to massage the insufflated region and expel the gas retained by the patient at the end of the procedure. The arc of rotation needed for the lever to engage the protruding knob 54a is labeled as 55. This locking position is not reached by the lever when the valve is opened by the insertion of the penetrator. The locking of the valve has to be done by the forceful and deliberate action of the surgeon. The small angle 52 shown at the bayonet locking stud 29 refers to the desirable slant for the groove 29 so as to insure that the locking force increases sufficiently to prevent accidental loosening between the proximal and the distal segments of the handle. The elasticity of the locking elements determiries the exact angle to be used, which should be somewhere between 2 and 5 degrees to account for tolerance errors. The infusion valve 11, its lever 11c, and its lever connector 11a are shown on FIG. 14. In FIG. 15, the opening of the valve is indicated by arrow 1d. FIG. 15 also shows a broken section of the valve shaft 34, its top “O” ring seal 34a, and its torsion spring 33 inserted into a slot in the operating bracket of valve 32. In the same FIG. 15, the seal 35 is seen, as well as the front surface 51a of the distal handle segment, which contacts the mating surface 51 of the proximal segment.

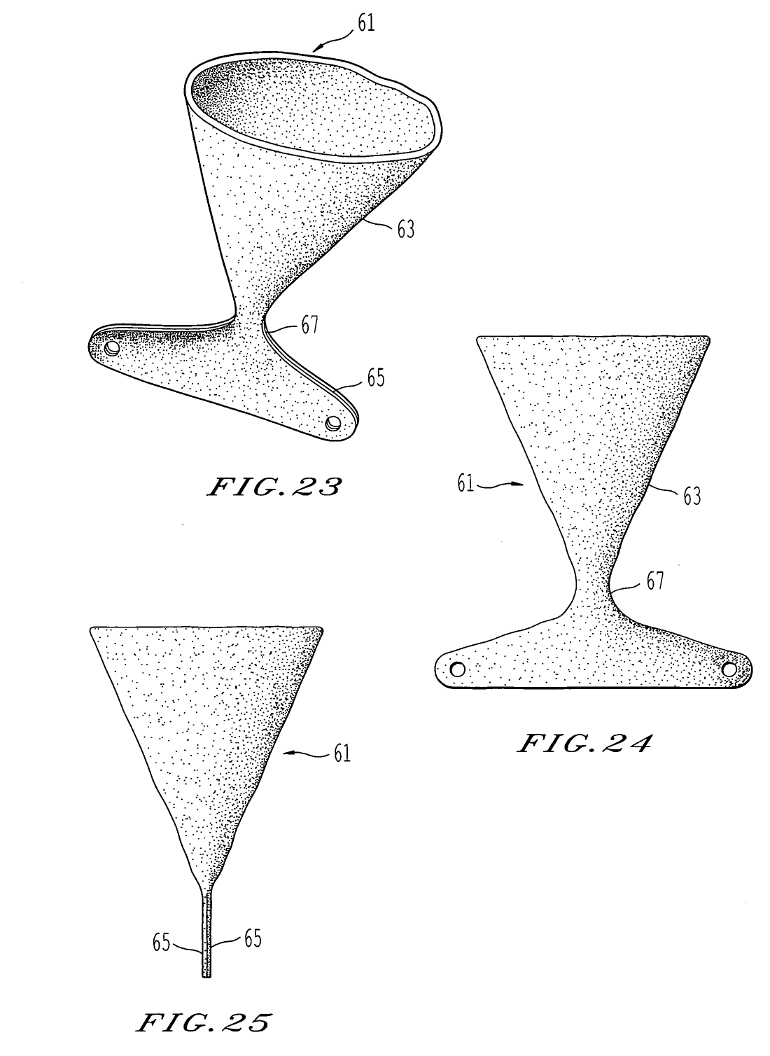

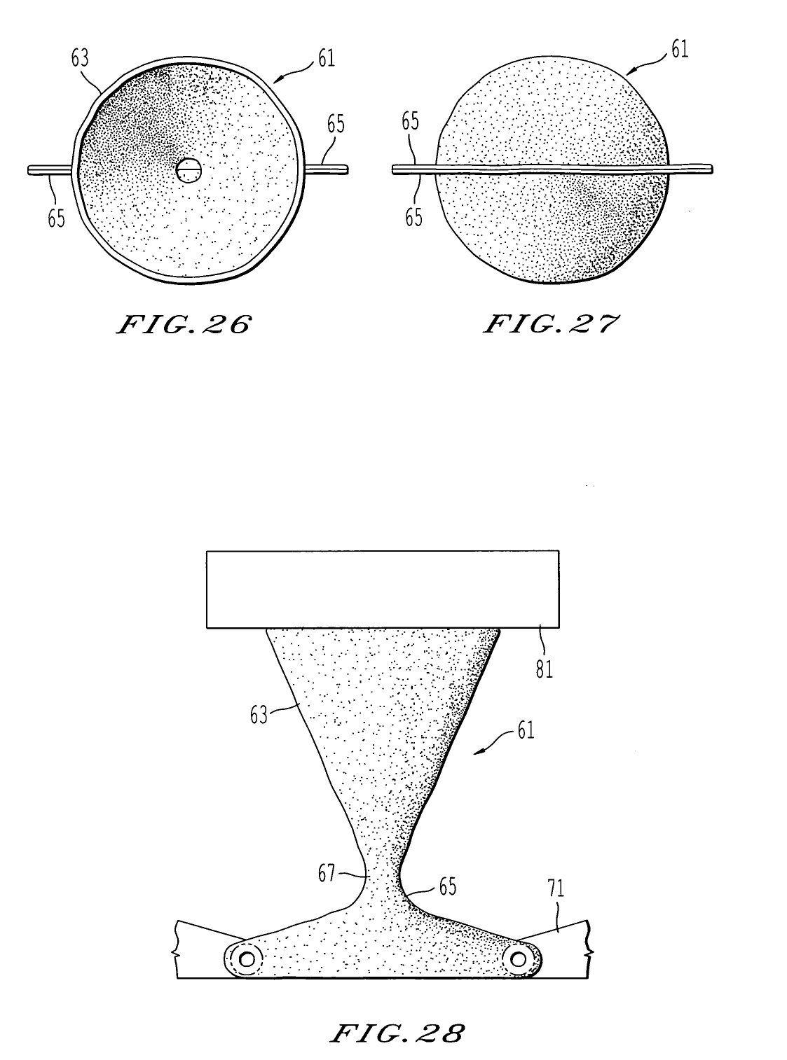

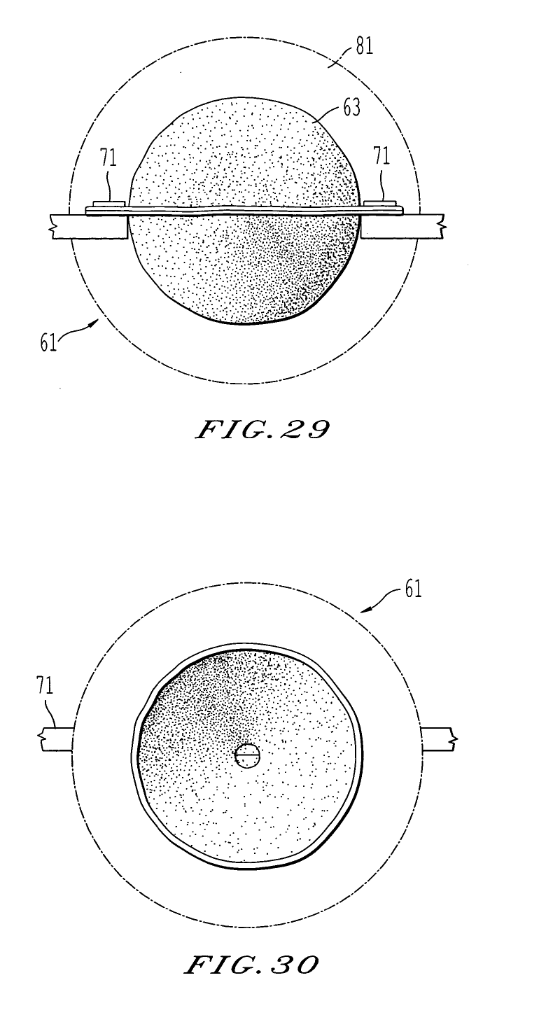

FIGS. 23-31 show an embodiment of a sealing member 61 that can maintain a gas tight seal within the trocar. The sealing member 61 can be used in place of the seal 35 and the flap valve 32 shown in FIG. 12, as well as the associated components for positioning and movement of the flap valve 32. Although the figures show preferred embodiments of the sealing member 61 disposed between the distal handle 6 and the penetrator 13 to maintain a gas tight seal therebetween, it is to be understood that the sealing member 61 can be disposed at any location within the trocar to maintain a gas tight seal, including between the distal handle 6 and any other instrument disposed within the trocar.

FIGS. 23-27 show isometric, front, side, top, and bottom views of the sealing member 61 in an uninstalled and unstretched or undeformed state, while FIGS. 28-31 show front, bottom, top, and side views of the sealing member 61 in an installed and stretched or deformed state in the distal handle 6.

The sealing member 61 can includes a conical portion 63, protrusions 65, and a neck portion 67 disposed between the conical portion 63 and the protrusions 65. When the sealing member 61 is installed in the distal handle 6, as shown in FIGS. 28-31, a top or larger diameter portion of the conical portion 63 can be disposed in a sealing ring 81 closer to the proximal handle 5 than a bottom or smaller diameter portion of the conical portion 63. The sealing ring 81 can be disposed in a void or other cooperating member in, or otherwise fastened to, the distal handle 6. The protrusion 65 can be stretched or deformed to be connected to attachment projections 71 secured to the inner wall of the distal handle 6, for example. The protrusions 65 can be in the form of flat flaps, such that deformation or stretching of the protrusions 65 urges the protrusions into contact with one other to form a gas tight seal. The protrusions 65 can define voids for fastening with the attachment projections 71. The neck portion 67 can be sized to form a gas tight seal with various instruments having various diameters. Preferably, components of the sealing member 61 are sufficiently elastic to provide a gas tight seal with various instruments having diameters between about 3 mm and about 12 mm.

By this arrangement, the sealing member 61 can permit insertion of the instrument (e.g., the penetrator 13), and can provide a gas tight seal therebetween by maintaining contact among components of the sealing member 61 and the instrument, and can provide a gas tight seal when no instrument is disposed in the sealing member 61 by maintaining contact among components of the sealing member 61. Specifically, contact can be maintained between the neck portion 67 and the instrument, can be maintained among the protrusions 65 and the instrument disposed in the sealing member 61, and/or can be maintained between the protrusions 65 when no instrument is disposed therein.

Applicant has discovered that the sealing member 61 can permit a large degree of relative motion and/or misalignment of the instrument disposed therein while maintaining a gas tight seal therebetween. Still further, because the sealing member 61 can maintain a gas tight seal when no instrument is disposed therein, the need for a separate flap valve (e.g., the flap valve 32), as well as the associated components of the flap valve, can be obviated. The sealing member 61 can be used when it is desired to prevent eversion or inversion of the sealing member, and can be used when it is desired to limit lateral movement of the sealing member and/or the instrument disposed in the sealing member.

In a preferred embodiment of the invention, portions of the sealing member 61 can be made of an elastic material, such as latex, silicone rubber, and/or SILASTIC™. The sealing member 61 can be cast in the shape shown in FIGS. 23-27. The sealing member 61 can be impregnated with a lubricant or otherwise lubricated (e.g., at the neck portion 67). Alternatively, the sealing member 61 can be formed or used without a lubricant.

Desufflation with the trocar including the sealing member 61 can be accomplished by removal of the gas line from the insufflation device 11, and venting gas through the open insufflation device 11. Applicants have determined that manual desufflation via hand pressure, which should be performed to force gas from the body as well as to prevent isolated gas pockets from remaining in the body, can be accomplished as effectively with the trocar including the sealing member 61 as with the trocar including the seal 35 and the flap valve 32.

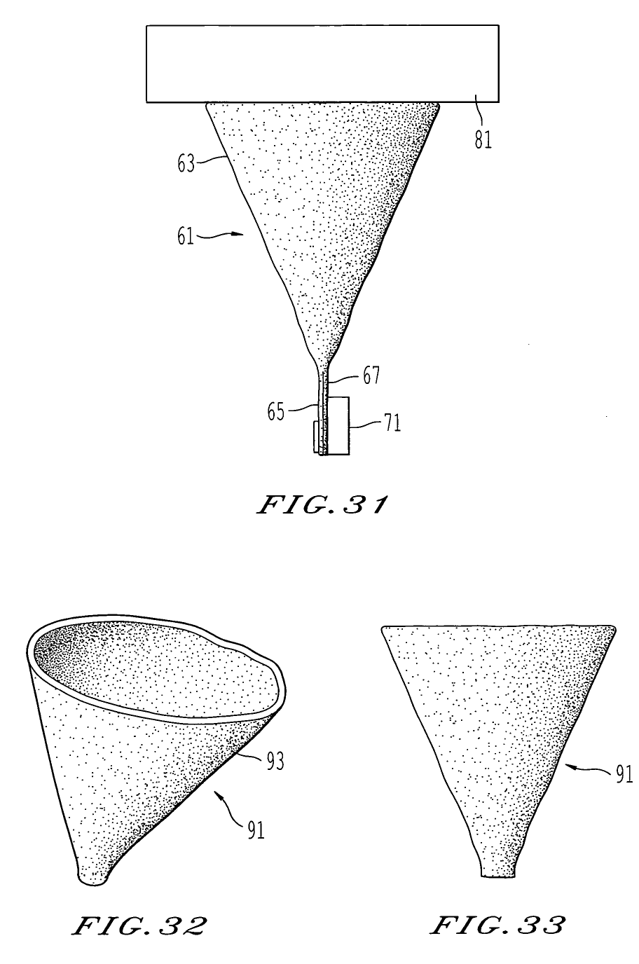

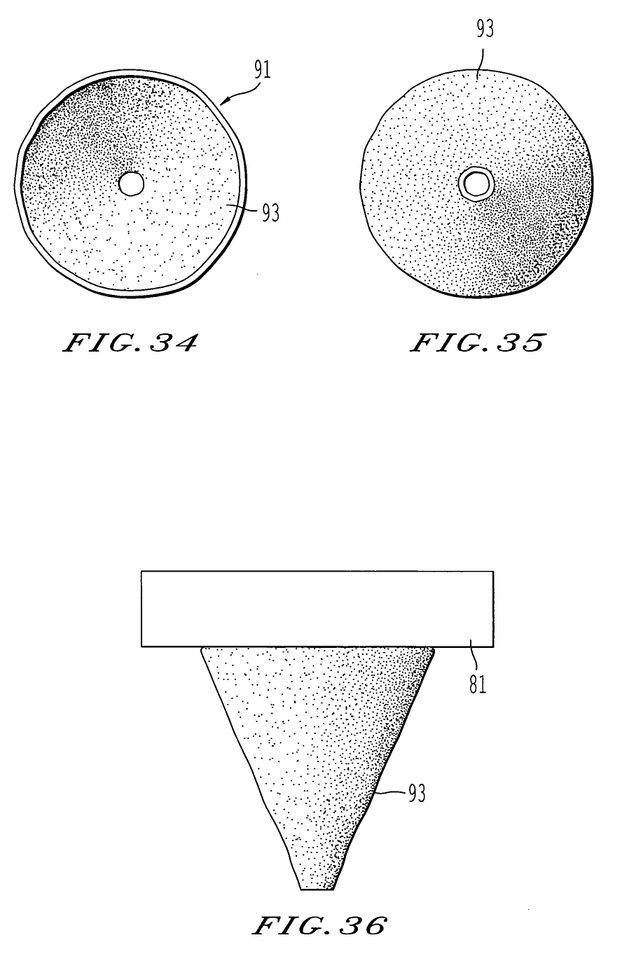

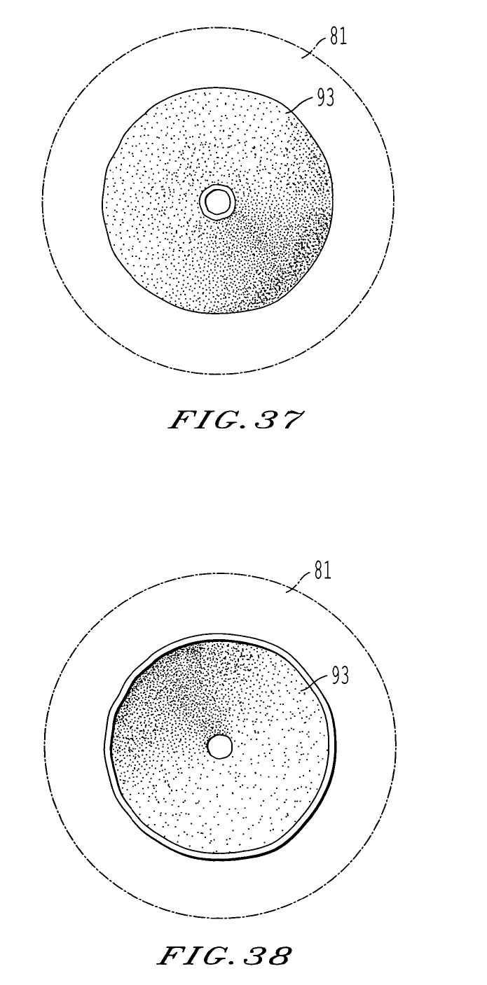

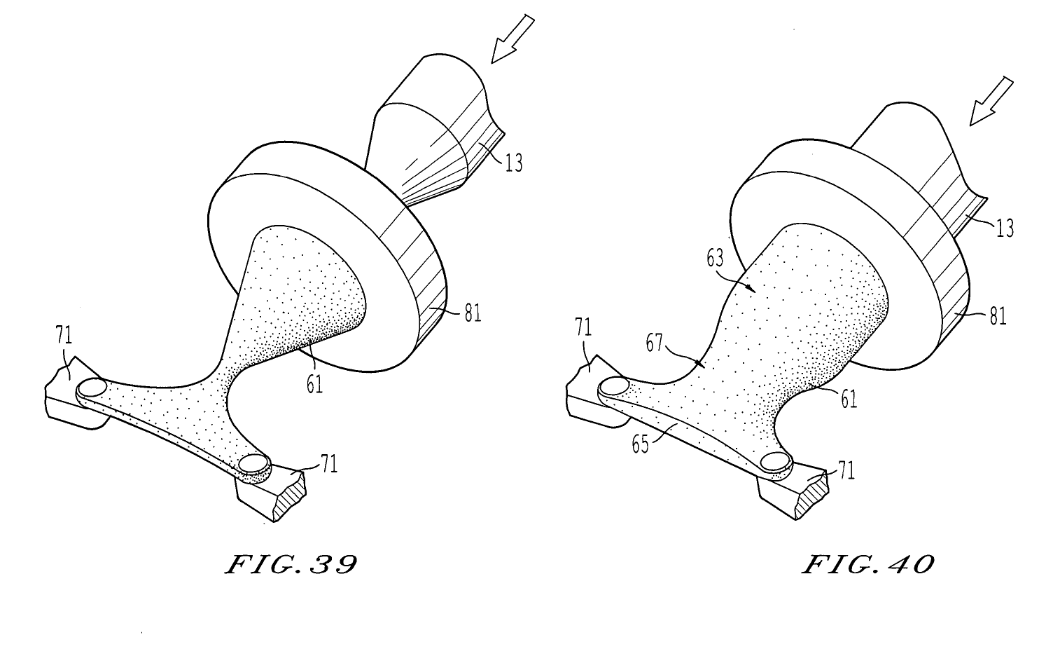

FIGS. 32-38 show an embodiment of a sealing member 91 that can maintain a gas tight seal within the trocar. The sealing member 91 can be used in place of the seal 35. Although the figures show preferred embodiments of the sealing member 91 disposed between the distal handle 6 and the penetrator 13 to maintain a gas tight seal therebetween, it is to be understood that the sealing member 91 can be disposed at any location within the trocar to maintain a gas tight seal, including between the distal handle 6 and any other instrument disposed within the trocar.

FIGS. 32-35 show isometric, front, top, and bottom views of the sealing member 91 in an uninstalled state, while FIGS. 36-38 show front, bottom, and top views of the sealing member 91 in an installed a state in the distal handle 6.

The sealing member 91 can includes a conical portion 91. When the sealing member 91 is installed in the distal handle 6, as shown in FIGS. 36-38, a top or larger diameter portion of the conical portion 93 can be disposed in a sealing ring 81 closer to the proximal handle 5 than a bottom or smaller diameter portion of the conical portion 93. The sealing ring 81 can be disposed in a void or other cooperating member in, or otherwise fastened to, the distal handle 6. Preferably, components of the sealing member 91 are sufficiently elastic to provide a gas tight seal with various instruments having diameters between about 3 mm and about 12 mm.

By this arrangement, the sealing member 91 can permit insertion of the instrument (e.g., the penetrator 13), and can provide a gas tight seal therebetween by maintaining contact among components of the sealing member 91 and the instrument. Specifically, contact can be maintained between the conical portion 93 and the instrument.

The conical portion 93 can include a height that is at least as large as a diameter of a base of the conical portion 93 before disposing the instrument within the sealing member 91. In a preferred embodiment, the height of the conical portion 93 is at least as large as a maximum diameter of the conical portion 93 before the instrument is disposed therein, and more preferably is larger than the maximum diameter of the conical portion 93 before disposing the instrument therein. Applicants have discovered that this arrangement can provide the sealing member 91 permitting a large degree of relative motion and/or misalignment of the instrument disposed therein while maintaining a gas tight seal therebetween. The sealing member 91 can be used when it is desired to permit eversion or inversion of the sealing member (e.g., when the instrument disposed therein is moved along a direction of withdrawal of the instrument from the sealing member 91), and can be used when it is desired to permit a larger range of lateral movement of the sealing member and/or the instrument disposed in the sealing member.

In a preferred embodiment of the invention, portions of the sealing member 91 can be made of an elastic material, such as latex, silicone rubber, and/or SILASTIC™. The sealing member 91 can be cast in the shape shown in FIGS. 32-35. The sealing member 91 can be impregnated with a lubricant or otherwise lubricated. Alternatively, the sealing member 91 can be formed or used without a lubricant.

Desufflation with the trocar including the sealing member 91 can be accomplished by removal of the gas line from the insufflation device 11, and venting gas through the open insufflation device 11, as well as by the conventional manner.

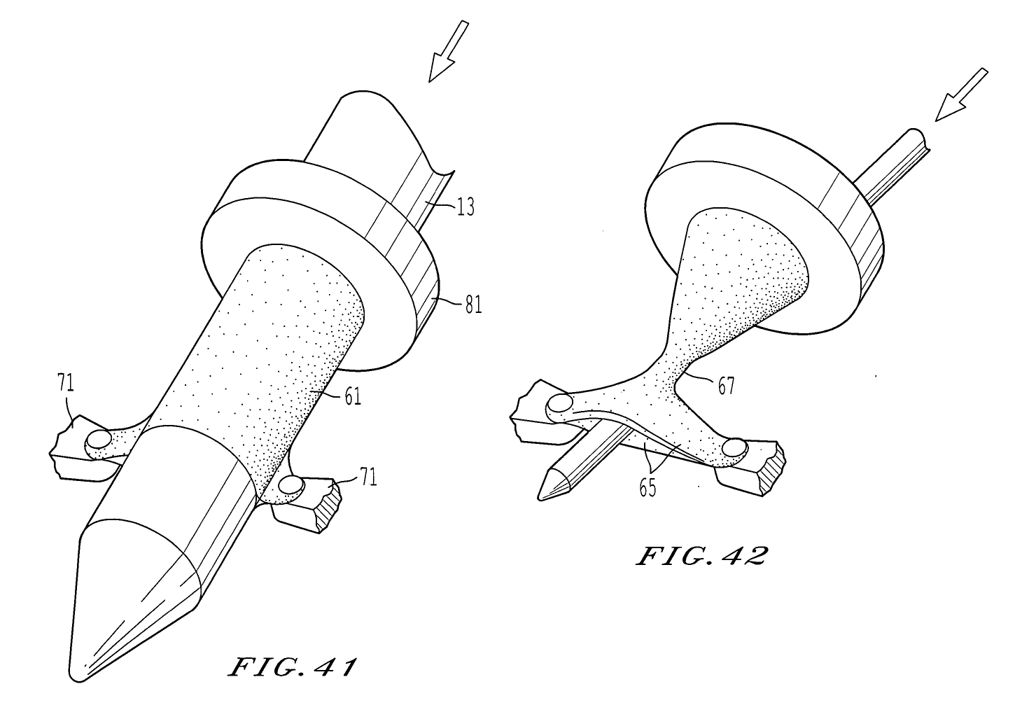

FIGS. 39-42 show examples of instruments disposed in the sealing member 61. Specifically, FIG. 39 shows an instrument (e.g., the penetrator 13) having a maximum diameter able to be disposed in the sealing member 61 partially disposed therein. The instrument is being urged into the sealing member 61 in the direction of the arrow. As shown in FIG. 40, as the instrument is further disposed in the sealing member 61, the conical portion 63 and the neck portion 67 can dilate to permit passage of the instrument through the sealing member 61 and can maintain a gas tight seal thereamong. As a result of the maximum dilation of the conical portion 63, the protrusions 65 can open partially. As shown in FIG. 41, after the instrument is fully disposed in the sealing member 61, the conical portion 63 and the neck portion 67 can be completely dilated. In this preferred embodiment, an initial minimum lumen of about 3 mm is increased to a maximum of about 12 mm. Dilation of the conical portion 63, the neck portion 67, and the protrusions 65 can limit axial motion and/or eversion/inversion of the sealing member 61 during one or more of pushing, twisting, and pulling of the instrument, as the sealing member 61 can be fastened to the attachment projections 71.

As shown in FIG. 42, the sealing member 61 can be used with an instrument of a minimum diameter. The neck portion 67 can expand a relatively smaller amount, but can continue to provide an effective gas tight seal. Further, misalignment between the sealing member 61 and the minimum diameter instrument will not result in a slot opening regardless of whether the instrument causes the neck portion 67 to be broadly displaced sideways, due to the relatively long length of the conical portion 61. In this preferred embodiment, the instrument has a diameter of about 4 mm and the neck portion has an initial minimum lumen of about 3 mm.

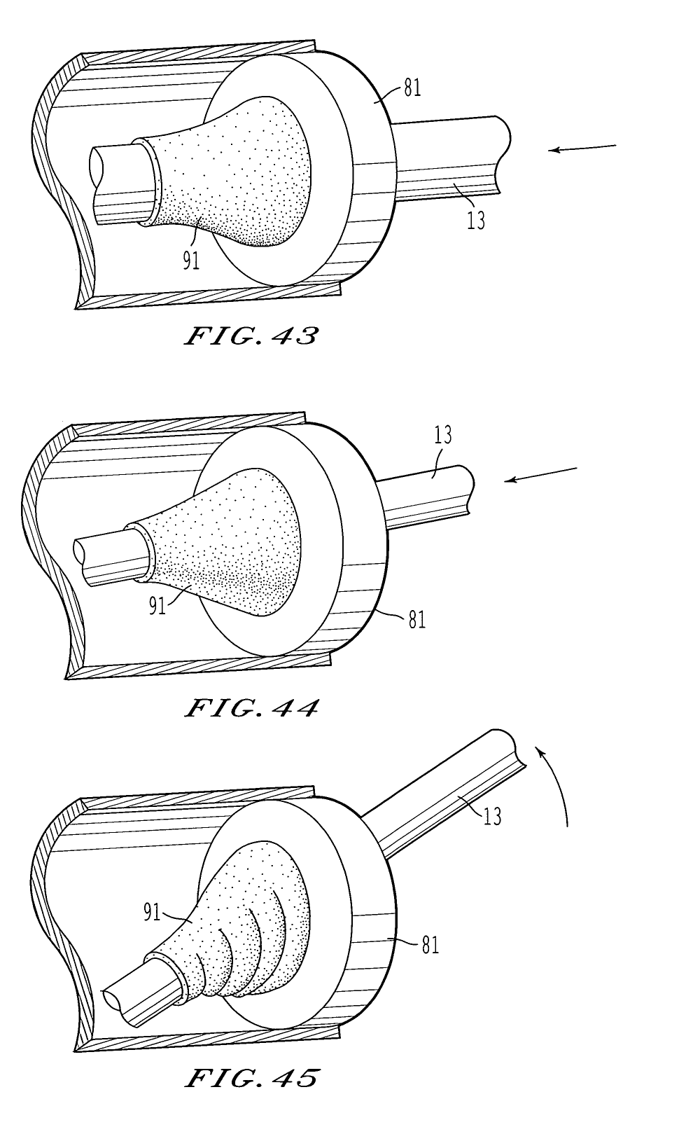

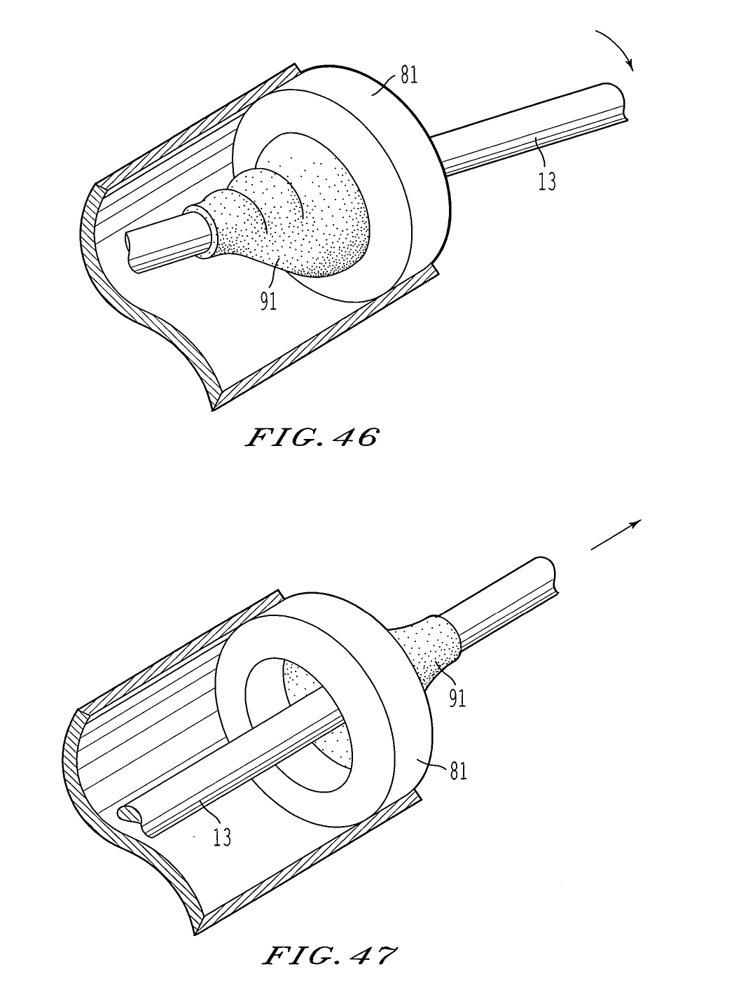

FIGS. 43-47 show examples of instruments disposed in the sealing member 91. Specifically, FIGS. 43 and 44 shows instruments (e.g., the penetrator 13 or any surgical instrument) fully disposed in the sealing member 91. The instrument is being disposed in the sealing member 91 in the direction of the arrow. The sealing member 91 can maintain a gas tight seal with both the larger and smaller diameter instruments. As shown in FIGS. 45 and 46, the sealing member 91 can permit relatively large lateral and angular misalignment (indicated by the arrows) between the sealing member 91 and the instrument disposed therein, and can maintain a gas tight seal therebetween.

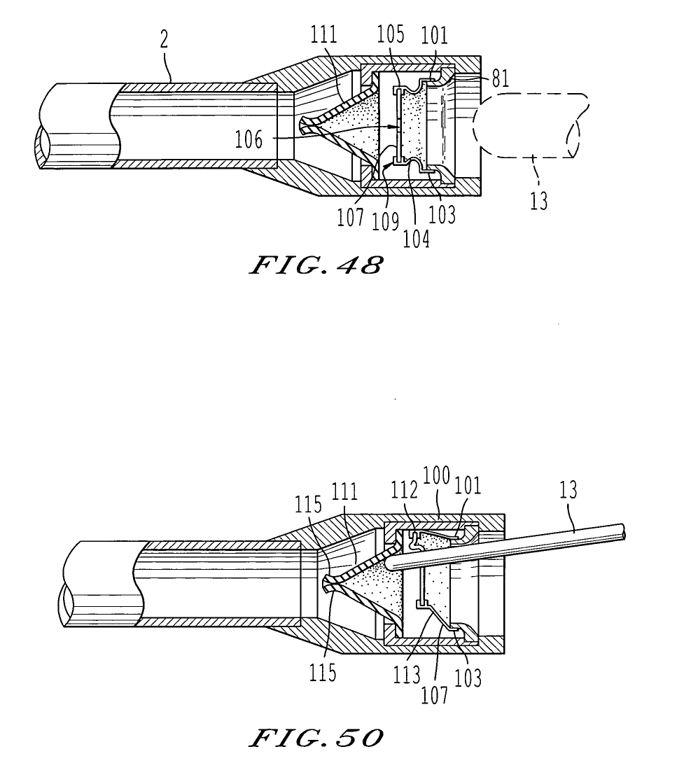

As shown in FIG. 47, when the instrument is retracted from the sealing member 91 by being moved in the direction of the arrow, the sealing member 91 can be everted/inverted, and the sealing member 91 can maintain a gas tight seal with the instrument throughout the period of retraction. After the instrument is fully removed from the sealing member 91, the sealing member can return to the initial non-everted position.

The seal and valve system proposed in the additional embodiment shown in FIGS. 48-51 is based on an elastomeric planar diaphragm 107, see FIG. 48, attached within a solid ring 109 and having a central orifice 106 of some three to four millimeters diameter. Such diaphragm 107, when made of the proper elastomeric material, will deflect radically to adapt to a wide variation of dilated lumens as may be required for adequate gas-tightness, however, it must also permit the frequent radial motion of the inserted instruments which accompany surgical procedures. Most seals of that type are affected by excessive radial deformation of the orifice, which is laterally elongated to an elliptical shape, causing very objectionable leakages of gas during critical surgical procedures.

In this invention such radial elliptical deformation of the diaphragm seal is completely eliminated within the desired range of application by a special elastic “floating mounting” consisting of an elastomeric tubular ring mounting 105 which surrounds the diaphragm mounting ring 109.

In this system, when the orifice is laterally displaced, and before it deforms sufficiently to allow gas leakage, the elastic mount tube 104 yields laterally and permits the radial displacement of the diaphragm to adapt to the lateral displacing instrument inserted, thereby maintaining a tight seal around the surface of contact with the instrument as will be shown later. FIG. 48 also shows a possible mounting within a cannula housing 2. In such Figure reference numeral 103 denotes the module housing containing the seal mounting ring 81 and the conoidal lip valve 111.

As shown in FIG. 48, the embodiment could be inserted into any suitable instrument whether surgical or industrial as a single unit for use under moderate pressures. The space between the diaphragm ring 109 and the base or flat end of the valve 111 is intended to allow the seal to move freely, twist, or move to the right or left within its space to permit accommodation to different instrument sizes; in other words, it floats within its space while limited only by the valve base at the left side and its mounting right 81 at the right side. As shown in FIG. 50, the elasticity of diaphragm 107 is such that the opening 106 can be stretched radially to be positioned immediately adjacent mounting ring 109.

The one-way valve 111 is an elastomeric surface of conoidal shape, meaning a surface connecting a line to a circle. The line is the contact between the sealing lips 115, 115 shaped to allow one-way operation by opening only towards the inside (left), while preventing gas leakage to the (right), or outside. The contacting lips of the seal edges should be preferably bent outside as shown in the drawings to reduce interference when complex instruments are drawn out across it.

The cannula 2 is shown as attached to the housing 6 (FIG. 1) as commonly done in this type of surgical instrument, but it is not the purpose here to consider that a limitation of this invention. The blunt cylindrical instrument shown as 13 in dotted lines in FIG. 48 represents the largest diameter size usable with the dimensions of the housing shown.

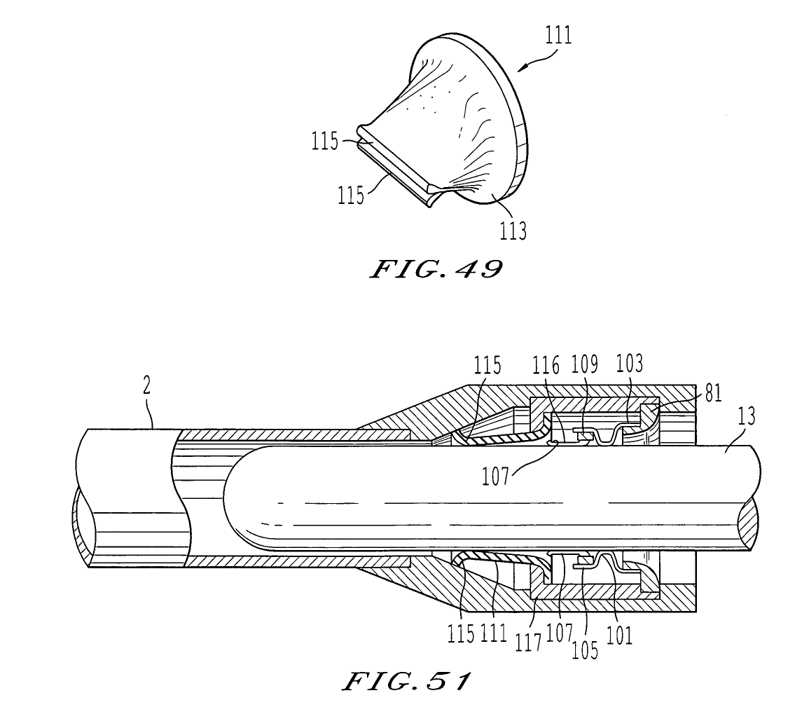

FIG. 49 shows the functions of the diaphragm floating seal when accommodating lateral displacements and instrument twists at the orifice. It shows an instrument of minimum size 13 as it enters across the seal completely along the edge of the inlet and at an angle; the worst possible case for a seal. The orifice has been displaced completely to one side. However, the floating tube 104 has bent sideways to minimize the strain across the orifice and allow a combination of orifice and diaphragm strain to permit the overall deformation without orifice opening. FIG. 49 shows the diaphragm contacting the inside of the housing at point 112, and while the diaphragm is shown compressed at the top, it is stretched at the bottom 113 there, but always keeping the sealing capabilities as desired. In practice the small diameter instrument 12 can be wiggled at will into the seal without the slightest leakage. A truly simple and inexpensive sealing for applications of this type.

FIG. 50 shows the conditions when the largest instrument 13 for this model size is inserted. As will be noticed, the seal orifice has been stretched to its limit by becoming completely adapted to the instrument outside surface as shown at area 116. The same can be seen where the conoid is shown stretched into a true cone by having its lips 115, 115 embrace the instrument cylindrical surface. The conoidal shape will be restored as the instrument is withdrawn and the sealing lips make contact in a straight line again.

To further explain the embodiment of FIGS. 48-51, such show an embodiment of a module housing 100 having a sealing member 101 and a valve 111 that can maintain a gas tight seal within the trocar. The sealing member 101 and the valve 111 can be used in place of the seal 35 and the flap valve 32 shown in FIG. 12, as well as the associated components for positioning and movement of the flap valve 32. Although the figures show preferred embodiments of the sealing member 101 and the valve 111 disposed between the distal handle 6 and the penetrator 13 or any surgical instrument to maintain a gas tight seal therebetween, it is to be understood that the sealing member 101 and/or the valve 111 can be disposed at any location within the trocar to maintain a gas tight seal, including between the distal handle 6 and any other instrument disposed within the trocar. It is further to be understood that the sealing member 101 and/or the valve 111, as is the case with the sealing members 61 and 91, are not limited to use in a trocar, and can be used to maintain a gas tight seal in a variety of industrial, mechanical, and/or electrical applications and be used on smaller diameter surgical devises such as Veress needle.

FIG. 48 shows a cross sectional view of the sealing member 101 and a valve 111. The sealing member 101 can include a first portion 103 configured to expand and/or contract a length of the first portion 103 along an axis thereof. The first portion 103 can be in the form of a bellows including one or more pleats that forms a floating tube 104. A top portion of the first portion 103 can be connected to a sealing or mounting ring 81. In a preferred embodiment, an interior of the top portion of the first portion 103 can be fastened to an exterior surface of the sealing ring 81. The sealing ring 81 can be disposed in a void or other cooperating member in, or otherwise fastened to, the distal handle 6.

A bottom portion of the first portion 103 can be connected to a second portion 105 of the sealing member 101. The second portion 105 can be configured to maintain a gas tight seal with an instrument disposed therein. The second portion 105 can include a diaphragm mounting ring 109 connected to a planar diaphragm 107 configured to form a gas tight seal with the instrument disposed in an opening 106 of the diaphragm 107, and can include a diaphragm ring 109 (FIG. 51) connecting the diaphragm 107 and the first portion 103. In a preferred embodiment, the diaphragm 107 can be fastened to the diaphragm ring 109, and an interior of the bottom portion of the first portion 103 can be fastened to an exterior of the diaphragm mounting ring 109. Preferably, components of the sealing member 101 are sufficiently elastic to provide a gas tight seal with various instruments having diameters between about 3 mm and about 12 mm, and a diameter of the opening of the diaphragm 107 can be between about 3 mm and about 4 mm when no instrument is disposed therein.

By this arrangement, the sealing member 101 can permit insertion of the instrument (e.g., the penetrator 13), and can provide a gas tight seal therebetween by maintaining contact among components of the sealing member 101 and the instrument. Specifically, contact can be maintained between the diaphragm 107 of the sealing member 101 and the instrument.

Applicants have discovered that the above arrangement can provide the sealing member 101 permitting a large degree of relative motion and/or misalignment of the instrument disposed therein while maintaining a gas tight seal therebetween. The sealing member 101 can be used when it is desired to permit a larger range of lateral movement of the sealing member and/or the instrument disposed in the sealing member. Specifically, the second portion 105 can be moved a relatively large amount relative to the trocar as a result of the connection of the second portion 105 to the first portion 103.

In a preferred embodiment of the invention, portions of the sealing member 101, such as the first portion 103 and/or the diaphragm 107, can be made of an elastic material, such as, for example, latex, silicone rubber, and/or SILASTIC™ or any other elastic material providing the elasticity desired. The sealing member 101, and more particularly the diaphragm 107 can be impregnated with a lubricant or otherwise lubricated. Alternatively, the sealing member 101 can be formed or used without a lubricant.

FIG. 49 shows an isometric view of the valve 111. As shown in the figure, the valve 111 can provide a gas tight seal between portions of the valve 111 when no instrument is disposed therein, and can be configured to permit flow between the instrument disposed therein and the valve 111, such that the valve 111 can act as a one way valve. The valve 111 can be in the form of a conoidal shape including a conical portion 113 and protrusions 115. In this context, conoidal is defined as a shape of a surface connecting a line and a circle. When the valve 111 is installed in the distal handle 6, a top or larger diameter portion of the conical portion 113 can be disposed in a valve ring 117 closer to the proximal handle 5 than a bottom or smaller diameter portion of the conical portion 113. The valve ring 117 can be disposed in a void or other cooperating member in, or otherwise fastened to, the distal handle 6. The protrusion 115 can be configured to permit gas flow in a first direction and to provide a gas tight seal in a second direction, such that the valve 11 can act as a one way valve. The protrusions 115 can be in the form of flat flaps urged into contact with one other to form a gas tight seal. End portions of the protrusion 115 can be disposed so as to extend in opposite directions away from a line of intersection of the protrusions 115, such that the end portions do not interfere with insertion and removal of the instrument. Preferably, components of the valve 111 are sufficiently elastic to accommodate various instruments having diameters between about 3 mm and about 12 mm, and to provide a gas tight seal when no instrument is disposed in the valve 111.

Desufflation with the trocar including the sealing member 101 and the valve 111 can be accomplished by removal of the gas line from the insufflation device 11, and venting gas through the open insufflation device 11. In an embodiment of the invention that uses the sealing member 101 and does not use the valve 111, desufflation can also be accomplished in the conventional manner.

FIGS. 50 and 50 show examples of instruments disposed in the sealing member 101, and in the sealing member 101 and the valve 111. Specifically, FIG. 50 shows a cross sectional view of a minimum diameter instrument (e.g. the penetrator 13) disposed in the sealing member 101, and FIG. 51 shows a cross sectional view of a maximum diameter instrument (e.g., the penetrator 13) disposed in the sealing member 101 and the valve 111. The sealing member 101 can maintain a gas tight seal with both the larger and smaller diameter instruments. As shown in FIGS. 50 and 51, the sealing member 101 can permit relatively large lateral and angular misalignment between the sealing member 101 and the instrument disposed therein, and can maintain a gas tight seal therebetween.

As shown in FIG. 50, when the instrument is not disposed in the valve 111, the protrusions 115 of the valve 111 provides a gas tight seal regardless of whether the instrument is disposed in the sealing member 101. The sealing member 101 can provide a gas tight seal with the instrument. As shown in FIG. 51, when the instrument is disposed in the sealing member 101 and the valve 111, the sealing member 1101 can provide a gas tight seal with the instrument. However, the valve 111 may be configured so as to not provide a gas tight seal with the instrument.

Numerous modifications and variations of the present invention are possible in light of the above teachings. It is therefore understood that within the scope of the appended claims, the invention may be practiced otherwise than as specifically described herein. In particular, it is understood that the present invention may be practiced by adoption of aspects of the present invention without adoption of the invention as a whole.

1. A surgical device, comprising:
a handle configured to be gripped;
a cannula connected to the handle; and
a sealing member disposed in an interior of the handle and configured to form a gas tight seal with an instrument disposed in an opening of the sealing member, the sealing member comprising:
a seal ring connected to the interior of the handle; and
a conical section configured to have the instrument disposed therein, the conical section being connected to the seal ring and having a height at least as large as a diameter of a base of the conical section before disposing the instrument therein.

2. The surgical device according to claim 2, wherein the conical section comprises an elastic material.

3. The surgical device according to claim 2, wherein the elastic material comprises one of silicone rubber and latex.

4. The surgical device according to claim 1, wherein the conical section comprises a first portion having a first diameter disposed adjacent the seal ring and a second portion having a second diameter less than the first diameter disposed opposite the first portion, and wherein the height of the conical section is at least as large as the first diameter before disposing the instrument in the conical seal.

5. The surgical device according to claim 4, wherein the height of the conical section is larger than the first diameter before disposing the instrument in the conical section.

6. The surgical device according to claim 1, wherein the height of the conical section is larger than the diameter of the base of the conical section before disposing the instrument in the conical section.

7. The surgical device according to claim 6, wherein the conical section comprises a first portion having a first diameter adjacent the seal ring and a second portion having a second diameter less than the first diameter opposite the first portion, and wherein the height of the conical section is larger than the first diameter before disposing the instrument in the conical section.

8. The surgical device according to claim 7, wherein the conical section is configured to be everted when the instrument is moved in an axial direction.

9. The surgical device according to claim 1, further comprising:
an instrument removably disposed in the conical section, the conical section forming the gas tight seal with the instrument.

10. The surgical device according to claim 9, wherein the instrument comprises a penetrator.

11. A surgical device, comprising:
a handle configured to be gripped;
a cannula connected to the handle; and
a sealing member disposed in an interior of the handle and configured to form a gas tight seal with an instrument disposed in an opening of the sealing member, the sealing member comprising:
a seal ring connected to the interior of the handle;
a conical section configured to have the instrument disposed therein, the conical section connected to the seal ring; and
first and second elastic protrusions connected to said conical section and configured to have the instrument disposed therein, the elastic protrusions configured to contact one another to form the gas tight seal.

12. The surgical device according to claim 11, wherein one of the conical section and the protrusions comprises an elastic material.

13. The surgical device according to claim 12, wherein the elastic material comprises one of silicone rubber and latex.

14. The surgical device according to claim 11, wherein the first and second protrusions comprise flaps.

15. The surgical device according to claim 14, wherein the flaps are stretched and connected to an interior of one of the cannula and the handle, such that the flaps are urged toward one another.

16. The surgical device according to claim 11, wherein the first and second protrusions comprises flat flaps configured to form the gas tight seal with the instrument when the instrument is disposed in the sealing member and configured to form the gas tight seal with one another when an instrument is not disposed in the sealing member.

17. The surgical device according to claim 11, wherein the sealing member comprises a neck disposed between the conical section and the protrusions, the neck configured to form the gas tight seal with the instrument disposed in the sealing member.

18. The surgical device according to claim 17, wherein the neck is configured to form the gas tight seal with the instrument having a diameter of between about 3 mm and about 12 mm disposed in the sealing member.

19. The surgical device according to claim 17, wherein the conical section comprises a first portion having a first diameter disposed adjacent the seal ring and a second portion having a second diameter less than the first diameter disposed adjacent the neck.

20. The surgical device according to claim 11, wherein the protrusions define voids, and one of the cannula and the handle comprises attachment portion connecting with the voids of the protrusions.

21. The surgical device according to claim 11, further comprising:
an instrument removably disposed in the sealing member.

22. The surgical device according to claim 21, wherein the instrument comprises a penetrator.

23. A sealing member configured to form a gas tight seal with an instrument removably disposed in a surgical device, the sealing member comprising:
a seal ring configured to be connected to the interior of the surgical device; and
a conical section connected to the seal ring and having a height at least as large as a diameter of a base of the conical section before the instrument is disposed in the seal.

24. The sealing member according to claim 23, wherein the conical section comprises an elastic material.

25. The sealing member according to claim 24, wherein the elastic material comprises one of silicone rubber and latex.

26. The sealing member according to claim 23, wherein the conical section comprises a first portion having a first diameter disposed adjacent the seal ring and a second portion having a second diameter less than the first diameter disposed opposite the first portion, and wherein the height of the conical section is at least as large as the first diameter before the instrument is disposed in the conical section.

27. The sealing member according to claim 26, wherein the height of the conical section is larger than the first diameter before the instrument is disposed in the conical section.

28. The sealing member according to claim 23, wherein the height of the conical section is larger than the diameter of the base of the conical section before the instrument is disposed in the conical section.

29. The sealing member according to claim 28, wherein the conical section comprises a first portion having a first diameter adjacent the seal ring and a second portion having a second diameter less than the first diameter disposed opposite the first portion, and wherein the height of the conical section is larger than the first diameter before the instrument is disposed in the conical section.

30. A sealing member configured to form a gas tight seal with an instrument removably disposed in a surgical device, the seal comprising:
a seal ring configured to be connected to an interior of the surgical device;
a conical section connected to the seal ring; and
first and second elastic protrusions configured to contact one another to form the gas tight seal.

31. The sealing member according to claim 30, wherein one of the conical section and the protrusions comprises an elastic material.

32. The sealing member according to claim 31, wherein the elastic material comprises one of silicone rubber and latex.

33. The sealing member according to claim 30, wherein the first and second protrusions comprise flaps.

34. The sealing member according to claim 33, wherein the flaps are configured to be urged toward one another when stretched.

35. The sealing member according to claim 30, wherein the first and second protrusions comprises flat flaps configured to form the gas tight seal with the instrument when the instrument is disposed in the seal and configured to form the gas tight seal with one another when an instrument is not disposed in the seal.

36. The sealing member according to claim 35, further comprising:
a neck disposed between the conical section and the protrusions, the neck configured to form the gas tight seal with the instrument disposed in the seal.

37. The sealing member according to claim 36, wherein the neck is configured to form the gas tight seal with the instrument having a diameter of between about 3 mm and about 12 mm disposed in the seal.

38. The sealing member according to claim 37, wherein the conical section comprises a first portion having a first diameter disposed adjacent the seal ring and a second portion having a second diameter less than the first diameter disposed adjacent the neck.

39. The sealing member according to claim 30, wherein the protrusions define voids configured to connect with attachment portions of the surgical device.

40. A surgical device, comprising:
a handle configured to be gripped;
a cannula connected to the handle; and
means for forming a gas tight seal between an instrument removably disposed therein, the means for forming the gas tight seal having a height at least as large as a diameter of a base of the means for forming the gas tight seal before disposing the instrument therein.

41. A surgical device, comprising:
a handle configured to be gripped;
a cannula connected to the handle; and
means for forming a gas tight seal between an instrument removably disposed therein and for forming the gas tight seal between portions of the means for forming the gas tight seal when no instrument is disposed therein.

42. A method of sealing a surgical device, comprising:
forming a seal between an instrunent and a sealing member, the sealing member having a height at least as large as a diameter of a base of the seal when the instrument is not disposed in the seal.

43. The method according to claim 42, further comprising:
disposing the sealing member in a handle of a trocar.

44. The method according to claim 42, wherein the instrument comprises a penetrator.

45. A method of sealing a surgical device, comprising:
disposing an instrument in a conical member; and
forming a seal between protrusions connected to the conical member and the instrument.

46. The method according to claim 45, wherein the protrusions comprises flaps.

47. The method according to claim 46, further comprising:
disposing the conical member and the protrusions in a handle of a trocar.

48. The method according to claim 46, wherein the instrument comprises a penetrator.

49. A method of sealing a surgical device, comprising:
disposing a sealing member in an interior of a handle; and
forming a gas tight seal with an instrument disposed in an opening of the sealing member, the sealing member comprising a seal ring connected to the interior of the handle, and a conical section configured to have the instrument disposed therein, the conical section connected to the seal ring and having a height at least as large as a diameter of a base of the conical section before disposing the instrument therein.

50. A method of sealing a surgical device, comprising:
disposing a sealing member in an interior of a handle; and
forming a gas tight seal with an instrument disposed in an opening of the sealing member, the sealing member comprising a seal ring connected to the interior of the handle, a conical section configured to have the instrument disposed therein, the conical section connected to the seal ring, and first and second elastic protrusions configured to have the instrument disposed therein, the elastic protrusions configured to contact one another to form the gas tight seal.

51. A surgical device, comprising:
a handle configured to be gripped;
a cannula connected to the handle; and
a sealing member disposed in an interior of the handle and configured to form a gas tight seal with an instrument disposed in an opening of the sealing member, the sealing member comprising:
a seal ring connected to the interior of the handle;
a first section connected to the seal ring; and
a second section connected to the first section and configured to have the instrument disposed therein.

52. The surgical device according to claim 51, wherein the first section comprises a bellows.

53. The surgical device according to claim 51, wherein the first section comprises a pleated section.

54. The surgical device according to claim 51, wherein the first section is configured to be extended along an axis of the sealing member.

55. The surgical device according to claim 51, wherein at least one of the first section and the second section comprises an elastic material.

56. The surgical device according to claim 55, wherein the elastic material comprises one of silicone rubber and latex.

57. The surgical device according to claim 51, further comprising:
an instrument removably disposed in the second section, the second section forming the gas tight seal with the instrument.

58. The surgical device according to claim 57, wherein the instrument comprises a penetrator.

59. The surgical device according to claim 51, further comprising:
a valve configured to form a gas tight seal when no instrument is disposed therein.

60. The surgical device according to claim 59, wherein the valve is configured to permit gas flow therethrough when the instrument is disposed therein.

61. The surgical device according to claim 59, wherein the valve comprises a one way valve.

62. The surgical device according to claim 59, wherein the valve comprises first and second protrusions configured to maintain the gas tight seal.

63. The surgical device according to claim 62, wherein the valve comprises a first cylindrical portion connected to the first and second protrusions.

64. A sealing assembly configured to form a gas tight seal with an instrument removably disposed in a device, the sealing assembly comprising:
a seal ring configured to be connected to an interior of the device;
a first section connected to the seal ring; and
a second section connected to the first section, the second section having an opening formed therein and being configured to permit the instrument to be disposed therethrough.

65. The sealing assembly according to claim 64, wherein the first section comprises a bellows.

66. The sealing assembly according to claim 64, wherein the first section comprises a pleated section.

67. The sealing assembly according to claim 64, wherein the first section is configured to be extended along an axis of the sealing member.

68. The sealing assembly according to claim 64, wherein at least one of the first section and the second section comprises an elastic material.

69. The sealing assembly according to claim 68, wherein the elastic material comprises one of silicone rubber and latex.

70. The sealing assembly according to claim 64, further comprising:
a valve configured to form a gas tight seal when no instrument is disposed therein.

71. The sealing assembly according to claim 70, wherein the valve is configured to permit gas flow therethrough when the instrument is disposed therein.

72. The sealing assembly according to claim 70, wherein the valve comprises a one way valve.

73. The sealing assembly according to claim 70, wherein the valve comprises first and second protrusions configured to maintain the gas tight seal.

74. The sealing assembly according to claim 73, wherein the valve comprises a first cylindrical portion connected to the first and second protrusions.

75. A method of sealing a device, comprising:
disposing a sealing member in an interior of the device, the sealing member having an opening therein and being configured to form a gas tight seal with an instrument disposed in the opening of the sealing member, the sealing member comprising a seal ring connected to the interior of the handle, a first section connected to the seal ring, and a second section connected to the first section and configured to have the instrument disposed therein.

76. The method according to claim 76, further comprising:
disposing a one way valve in an interior of the device, the valve configured to achieve a gas tight seal when no instrument is disposed in the valve.
